SEQUENCE LISTING (12 sequences)

SEQ ID NO: 1 - DNA, 21 bp, Artificial Sequence
  Synthetic - Primer
accaggaactaatcagacaag

SEQ ID NO: 2 - DNA, 25 bp, Artificial Sequence
  Synthetic - Primer
gacttgatctttgaaatttggatct

SEQ ID NO: 3 - DNA, 42 bp, Artificial Sequence
  Synthetic - Primer
ttccgaagaacgctgaagcggaactgattacaaacattggcc

SEQ ID NO: 4 - DNA, 40 bp, Artificial Sequence
  Synthetic - Primer
cgcattggcatggaagtcacaatttgatggcacctgtgta

SEQ ID NO: 5 - DNA, 21 bp, Artificial Sequence
  Synthetic - Primer
gggggcaaattgtgcaatttg

SEQ ID NO: 6 - DNA, 20 bp, Artificial Sequence
  Synthetic - Primer
cttcgggaacgtggttgacc

SEQ ID NO: 7 - DNA, 18 bp, Artificial Sequence
  Synthetic - Primer
cggtggacaaattgtcac

SEQ ID NO: 8 - DNA, 22 bp, Artificial Sequence
  Synthetic - Primer
cttctctggatttaacacactt

SEQ ID NO: 9 - DNA, 51 bp, Artificial Sequence
  Synthetic - Primer
tcagcacacaaagccaaaaatttatttttctgtgcaaaggaaattaaggag

SEQ ID NO: 10 - DNA, 49 bp, Artificial Sequence
  Synthetic - Primer
tattggtggagctaaacttaaagccttttctgtacaatccctttgagtg

SEQ ID NO: 11 - DNA, 28 bp, Artificial Sequence
  Synthetic - Primer
ttacaagcttaaagaatgtctgaacact

SEQ ID NO: 12 - DNA, 27 bp, Artificial Sequence
  Synthetic - Primer
ttgaatttaggtgaaacatttgtcacg

(19) United States
(12) Patent Application Publication
(10) Pub. No.: US 2024/0124945 A1
(43) Pub. Date: Apr. 18, 2024

(54) SYSTEMS AND METHODS FOR DETECTING MICROORGANISM OR VIRAL LOADED AEROSOLS

(71) Applicant: Massachusetts Institute of Technology, Cambridge, MA (US)

(72) Inventors: Domitilla Del Vecchio, Cambridge, MA (US); Hsin-Ho Huang, Arlington, MA (US); Carlos Barajas, Cambridge, MA (US); Kalon J. Overholt, Somerville, MA (US); Simone Bruno, Cambridge, MA (US); Theodore W. Grunberg, Cambridge, MA (US)

(21) Appl. No.: 18/277,268

(22) PCT Filed: Feb. 16, 2022
(86) PCT No.: PCT/US2022/016667

§ 371 Date: Aug. 15, 2023

Related U.S. Application Data
(60) Provisional application No. 63/149,756, filed on Feb. 16, 2021.
(60) Provisional application No. 63/285,200, filed on Dec. 2, 2021.

(51) Int. Cl. C12Q 1/70; B01L 3/00; B01L 7/00; C12Q 1/04; G01N 1/40; G01N 15/1434

(52) U.S. Cl. CPC C12Q 1/70; B01L 3/502761; B01L 7/52; C12Q 1/04; G01N 1/40; G01N 15/1436; G01N 2015/011

(57) ABSTRACT

A collecting system is provided that can include a probe configured to collect pathogens from a surrounding fluid, an elution chamber containing a liquid solvent and configured to receive the probe to elute the pathogens collected on the probe using the liquid solvent, and a heater configured to lyse the pathogens to release the genetic material of the pathogens into the liquid solvent.

### CROSS-REFERENCE TO RELATED APPLICATIONS

This application claims priority to U.S. patent application Ser. No. 63/149,756 filed Feb. 16, 2021, and entitled, “A System for On-Site Detection of Virus-Loaded Aerosol,” and U.S. patent application Ser. No. 63/285,200 filed Dec. 2, 2021, and entitled, “A System for On-Site Detection of Virus-Loaded Aerosol,” each of which is hereby incorporated by reference in its entirety.

### STATEMENT REGARDING FEDERALLY SPONSORED RESEARCH

This invention was made with Government support under Grant No. FA9550-20-1-0044 awarded by the Air Force Office of Scientific Research. The Government has certain rights in the invention.

### BACKGROUND

Pathogen testing of sick individuals typically involves swabbing of internal regions of a patient (e.g., the nasal passage) to collect these potential pathogens (e.g., assuming the patient is harboring these potential pathogens). The swab is then dispersed in a solvent to disperse the pathogen in the solvent. After, the pathogen laden solvent (or the solvent being free of the pathogen) can be subjected to any number of tests for a specific pathogen including, for example, a polymerase chain reaction (“PCR”) test, an antigen based test (e.g., with the antigen being derived from the pathogen of interest), etc. These tests can confirm whether or not the patient has a sufficient amount of the specific pathogen. If the test confirms that the patient has the specific pathogen, the patient can be instructed to quarantine, or otherwise avoid close contact with other individuals to prevent spreading of the pathogen from the tested individual to other individuals.

Recently with the severe acute respiratory syndrome coronavirus 2 (“SARS-CoV-2”), the separation between individuals that can and cannot transmit the virus has become muddied. For example, a significant percentage of the individuals inflicted with SARS-CoV-2 are asymptomatic, but can transmit the virus. Thus, these asymptomatic individuals who are unlikely to seek conventional testing methods can actively transmit the virus to other individuals. In addition, the possible believed transmission mechanisms for SARS-CoV-2 have frequently changed since the discovery of SARS-CoV-2. For example, at the beginning, SARS-CoV-2 was thought to transmit by contact with surfaces (e.g., it was asserted that the SARS-CoV-2 could “live” on surfaces for hours to days), however, additional data suggested that, rather the SARS-CoV-2 could aerosolize and thus could be transmitted through the air. This inability to accurately identify the actual viral transmission mechanisms, forced individuals to improperly weigh the risks of transmission.

The aerosolization mechanism of the SARS-CoV-2 shifted the thinking about how to minimize transmission of a pathogen that can aerosolize. For example, currently, the only way to definitely know whether or not an enclosed volume is safe is to test every single individual for the pathogen (e.g., by swabbing them), which is logistically impractical. However, even this may not completely mitigate all risk. For example, in the case of an aerosolizable pathogen, the flux of individuals into (and out of) the enclosed volume can cause the aerosolized pathogen to linger in the air—even if every current individual within the enclosed volume is not transmitting the pathogen. In other words, testing every individual still does not prevent an infected individual from dispersing pathogen laden aerosols within the enclosed volume. Thus, it would be desirable to have improved systems and methods for detecting microorganism or viral loaded aerosols.

### SUMMARY OF THE DISCLOSURE

Some non-limiting examples of the disclosure provide a collecting system. The collecting system can include a probe that can be configured to collect pathogens from a surrounding fluid, an elution chamber containing a liquid solvent and that can be configured to receive the probe to elute the pathogens collected on the probe using the liquid solvent, and a heater that can be configured to lyse the pathogens to release the genetic material of the pathogens into the liquid solvent.

In some embodiments, the collecting system can include an elution device configured to elute the pathogens collected on the probe into the liquid solvent. The elution device can include a container that defines the elution chamber, and a heat block in thermal communication with the heater and the elution chamber. The heater can be configured to heat the heat block thereby heating the liquid solvent to lyse the pathogens.

In some non-limiting examples, a collecting system can include an elution device configured to elute the pathogens collected on a probe into a liquid solvent. The elution device can include at least one of a mixer that mixes the liquid solvent and forces the liquid solvent into contact with the probe to elute the pathogens off the probe and into the liquid solvent, an electrode configured to store charge and repel or attract the pathogens or the genetic material of the pathogens away from the probe and into the liquid solvent; or a power source in selective electrical communication with the probe, the power source being configured to charge the probe to repel the pathogens or the genetic material of the pathogens away from the probe into the liquid solvent.

In some non-limiting examples, a collecting system can include a concentrator configured to concentrate the genetic material of the pathogens.

In some non-limiting examples, a concentrator can include a semi-permeable barrier positioned within a flow path of genetic material of pathogens, and an electrode that can be configured to be positively or negatively charged to direct the genetic material of the pathogens towards the semi-permeable barrier to concentrate the genetic material.

In some non-limiting examples, a concentrator can include a first electrode that can be configured to be positively charged. A concentrator can include a second electrode that can be configured to be negatively charged. A semipermeable barrier can be positioned between the first electrode and the second electrode.

In some non-limiting examples, a collecting system can include at least one of a robotic arm that can be configured to selectively grasp a probe to place the probe into and out of an elution chamber, or an actuator that is coupled to the probe. The actuator can be configured to be extended to place the probe into the elution chamber or retracted to remove the probe from the elution chamber.

In some non-limiting examples, a collecting system can include a detection system that can be configured to detect a presence of pathogens.

In some non-limiting examples, a detection system can include a detection chamber, a light source optically coupled to the detection chamber, and a photodetector optically coupled to the detection chamber.

In some non-limiting examples, a collecting system can be configured to amplify an amount of genetic material.

In some non-limiting examples, a detection system can be configured to detect a presence of a pathogen by detecting a genetic material of lysed pathogens.

In some non-limiting examples, a collecting system can include a computing device. The computing device can be configured to determine that the pathogen is aerosolizable based on the presence of the pathogens having been detected. The pathogen having been previously unknown of being aerosolizable. The computing device can be configured to notify a user based on the determination that the pathogen is aerosolizable.

In some non-limiting examples, genetic material can be amplified without the use of a thermocycler.

In some non-limiting examples, a collecting system does not use a thermocycler.

In some embodiments, a collecting system can include a thermocycler that is configured to cycle the temperature of the liquid solvent and the genetic material to amplify the genetic material.

In some embodiments, a collecting system can include one or more reagents that are configured to implement a PCR on the genetic material to amplify the genetic material.

In some embodiments, at least one of one or more cycles that define a PCR take less than 120 minutes, or the one or more reagents, a thermocycler, or both are configured to implement a fast PCR on genetic material to amplify the genetic material. The one or more cycles that define the fast PCR can be less than 3 minutes. The one or more cycles can be at least one of greater than 30 cycles, greater than 35 cycles, or greater than 40 cycles.

In some non-limiting examples, a collecting system can include a computing device in communication with the detection system. The computing device can be configured to cause the light source to emit first light towards the detection chamber. The first light can interact with a molecule that interacts with the genetic material of the pathogens to emit second light. The computing device can be configured to receive optical data from the photodetector indicative of the second light interacting with the photodetector, and determine a presence of the pathogen, based on the optical data.

In some non-limiting examples, a collecting system can be configured to at least one of implement a reverse transcription loop mediated isothermal amplification (RT-LAMP) reaction on genetic material from a pathogen to amplify the amount of genetic material, or implement a reverse transcription polymerase chain reaction (“RT-PCR”) on the genetic material from the pathogen to amplify the amount of genetic material.

In some non-limiting examples, a collecting system can include one or more reagents that can include a primer specific to a corresponding region on the genetic material of the pathogen, a reverse transcriptase, and a deoxyribonucleic acid (“DNA”) polymerase.

In some non-limiting examples, a collecting system can include one or more reagents that can be configured to implement the RT-LAMP reaction. The one or more reagents can be preloaded within the detection chamber.

In some non-limiting examples, one or more reagents can be lyophilized.

In some non-limiting examples, a heater or another heater can be configured to heat genetic material and one or more reagents to a temperature that is greater than or equal to 65° C.

In some non-limiting examples, genetic material can be amplified by using a fast thermocycler.

In some non-limiting examples, a collecting system uses a fast thermocycler.

In some non-limiting examples, a collecting system can be configured to implement a fast reverse transcription polymerase chain reaction (fast RT-PCR) on genetic material from a pathogen.

In some non-limiting examples, a collecting system can include one or more reagents that can be configured to implement the fast RT-PCR. The one or more reagents can be preloaded within the detection chamber.

In some non-limiting examples, a thermocycler can be configured to heat and cool genetic material and one or more reagents to a temperature that is between 60° C. and 95° C.

In some non-limiting examples, a computing device can be configured to determine an initial amount of the genetic material in the detection chamber before amplification, based on optical data, and determine a concentration of the pathogen in the enclosed volume, based on the initial amount of genetic material and a total volume of the enclosed volume.

In some non-limiting examples, a computing device can be configured to determine a concentration of a pathogen in an enclosed volume, based on an initial amount of genetic material, a total volume of the enclosed volume, and an efficiency ratio of a collection rate of the pathogens in the surrounding volume to the pathogens collected on a probe.

In some non-limiting examples, a collecting system can include a pump that can be configured to move the genetic material from the elution chamber and to the detection chamber.

In some non-limiting examples, a collecting system can include a power source in selective electrical communication with a probe. The probe can be in selective electrical communication with an electrical ground. When a probe is electrically connected to the electrical ground, the probe can attract and collect aerosolized pathogens that have been ionized. When the probe is electrically connected to the power source, the power source can charge the probe to repel the pathogens or the genetic material of the pathogens away from the probe into the liquid solvent.

In some non-limiting examples, a volume of a liquid solvent can be at least one of less than or equal to 250 μL, less than or equal to 150 μL, less than or equal to 100 μL, less than or equal to 50 μL, less than or equal to 25 μL, or less than or equal to 12.5 μL.

Some embodiments of the disclosure provide a method for collecting and analyzing particles. The method can include collecting particles on a probe, placing the probe into an elution chamber that includes a liquid, lysing the particles while the probe is positioned within the elution chamber, so that the particles release analytes within the liquid thereby creating analyte solution, concentrating the analyte solution to create a concentrated analyte solution, and detecting the analyte to detect the presence of the particles.

In some embodiments, lysing particles can include heating liquid with a probe positioned within an elution chamber thereby lysing the particles to release analytes.

In some embodiments, analyte can be charged. A method can include electrically charging a probe to repel the analyte away from the probe.

In some embodiments, an analyte can be charged. Concentrating the analyte includes charging one or more electrodes to cause the analyte to concentrate into a concentrated analyte solution.

In some embodiments, particles can be a type of pathogen. An analyte can be genetic material of the type of pathogen.

In some embodiments, a type of pathogen can be a virus. Genetic material can be a single stranded RNA.

Some non-limiting examples of the disclosure provide a bioaerosol amplification and detection system for continuous monitoring of an indoor environment to detect airborne viral particles. The system can include a mobile and autonomously moving aerosol sampler unit comprising a housing and a probe exposed to air and attached to the housing. Airborne viral particles can collect on the collector probe. The system can include a bioaerosol analysis platform configured to amplify and detect the airborne viral particles collected on the collector probe by one or more airborne viral particle detection assays. The platform can include a miniature vortex mixer comprising an extraction tube containing an aqueous solution, an nucleic acid extraction chamber, a fluid transfer system, and a nucleic acid detection chamber. The bioaerosol analysis platform can be connected to the mobile aerosol sampler unit and the bioaerosol analysis platform can provide detection output at the site of monitoring.

In some non-limiting examples, airborne particles can collect on a probe based on corona discharge ionization and electrostatic precipitation.

In some non-limiting examples, collected airborne particles can enter the analysis platform after being detached from the probe.

In some non-limiting examples, an aqueous solution is deionized water or an electrically conductive buffer.

Some non-limiting examples of the disclosure provide a method of detecting airborne viral particles in an indoor environment and classifying the level of particles as being above or below a pre-determined acceptable level. The method can include collecting airborne viral particles, detecting collected amplified viral particles or components with one or more detection assays, and providing detection output in near-real time at the site of detection to classify the level of particles as being above or below the pre-determined acceptable level.

In some non-limiting examples, one or more detection assays can be a nucleic acid detection.

In some non-limiting examples, a nucleic acid detection can be performed in a one-step reaction without the use of a thermocycler.

In some non-limiting examples, a nucleic acid detection is RT-LAMP.

In some non-limiting examples, a nucleic acid detection can be performed in a multi-step reaction with the use of a fast thermocycler.

In some non-limiting examples, a nucleic acid detection is fast RT-PCR.

In some non-limiting examples, particles can be first collected on a probe and the particles can be then removed from the probe and collected in a tube filled with aqueous solution.

In some non-limiting examples, collected particles can be detached to aqueous solution using a vortex mixer.

In some non-limiting examples, a vortex mixer can be miniaturized to fit a single 2 mL tube.

In some non-limiting examples, a volume of the aqueous solution in the collection tube can be between 50-250 μL.

In some non-limiting examples, a limit of detection of viral particles can be less than or equal to 50 particles per 25 μl. In some non-limiting examples, a limit of detection of viral particles can be less than or equal to 5 particles per 25 μl.

In some non-limiting examples, a detection is by a one-step chemical reaction with a fluorescent readout.

In some non-limiting examples, if a fluorescent readout level is above a pre-set threshold within a given amount of time, then a system can output a first signal at the site of monitoring to denote a dangerous level of viral particle. If the fluorescent readout level is below the pre-set threshold within the given amount of time, then the system can output a signal different from the first signal to denote that the level of viral particles in the air is not at a dangerous level.

Some non-limiting examples of the disclosure provide a mobile detection device for detecting airborne viral particles. The device can include a metal probe configured to collect viral particles from air, and a system that can include a tube configured to receive the probe after sampling, a heating element configured to heat a first end of the tube, a fluid transfer system comprising a pump connected to the tubing proximate the first end, a concentrator, a RT-LAMP reaction chamber including a heated shell configured to maintain a temperature in the RT-LAMP reaction chamber, and a readout system comprising a light source and a detector configured to monitor the RT-LAMP reaction chamber for fluorescence.

In some non-limiting examples, a heated tubing can be regulated to be between 75-125° C.

In some non-limiting examples, a heated tubing can be regulated to be at about 90° C.

In some non-limiting examples, a RT-LAMP reaction chamber can be held at a constant temperature in the range of 50-80° C.

In some non-limiting examples, a temperature can be held constant at 65° C.

Some non-limiting examples of the disclosure provide a mobile detection device for detecting airborne viral particles. The device can include a metal probe configured to collect viral particles from air, and a system that can include a tube configured to receive the probe after sampling, a heating element configured to heat a first end of the tube, a fluid transfer system comprising a pump connected to the tubing proximate the first end, a concentrator, a fast RT-PCR chamber including a heated shell configured to cycle the temperature in the fast RT-PCR chamber, and a readout system comprising a light source and a detector configured to monitor the fast RT-PCR chamber for fluorescence.

In some non-limiting examples, a fast RT-PCR chamber can be cycled for one or more cycles between the temperatures of 60° C. and 95° C.

In some non-limiting examples, a temperature can be held between 60° C. and 95° C.

In some non-limiting examples, a tube can have a volume between 1.5 and 2.5 mL.

In some non-limiting examples, a tube can have a volume of about 2 mL.

Some non-limiting examples of the disclosure provide a method of detecting the presence of airborne viral particles in an indoor environment and classifying the level of particles as being above or below a pre-determined acceptable level. The method can include collecting airborne viral particles, detecting collected amplified viral particles or components with one or more detection assays, and providing detection output in near-real time at the site of detection to classify the level of particles as being above or below the pre-determined acceptable level.

Some non-limiting examples of the disclosure provide a method of detecting the presence of airborne viral particles in an indoor environment. The method can include capturing viral particles on a metal collector probe and detaching nucleic acids from the viral particles on the probe using electrostatics that can include collecting airborne viral particles on a metal surface probe via electrostatic precipitation, the viral particles remaining adsorbed to the surface probe when it is brought into contact with an aqueous solvent, extracting nucleic acids from the viral particles via heating, causing virions to lyse their genomic content in the vicinity of the probe surface; applying a negative surface charge to the probe using a capacitor, such that nucleic acid is repelled from the surface and nucleic acid molecules distribute into a bulk aqueous solution; and moving the extracted and captured nucleic acid into a smaller concentrated volume for subsequent biomolecular analysis. The analysis can include injecting the bulk aqueous solution into a capillary, introducing a voltage differential between two electrodes present in their own chambers on either end of the capillary, allowing nucleic acid to migrate via electrophoresis until it reaches a semipermeable membrane, which stops it from traveling further, wherein a concentration gradient of nucleic acid develops against the semipermeable membrane, after the gradient has developed, closing a valve to isolate the most concentrated fraction of the nucleic acid and create an nucleic acid-enriched solution; and directing the nucleic acid-enriched solution via a pump into another chamber where a biomolecular assay is performed.

In some non-limiting examples, an concentrator can be used to enrich the concentration of nucleic acids.

In some non-limiting examples, an enrichment device can include an agarose block on one end and a semipermeable membrane on the other end.

In some non-limiting examples, an enrichment device can include a polydimethylsiloxane (PDMS) chip forming a base and sides of a channel with two ends, semipermeable membranes sealing both ends of the channel, 2% agarose gel blocks immediately outside of the membranes to prevent convection, and a glass plate forming the top of the channel.

In some non-limiting examples, a channel can include a solution that can be either 5.4 or 0.6 ng/μL nucleic acid in 0.125% agarose and Tris-acetate-EDTA (TAE) buffer.

In some non-limiting examples, an amount of nucleic acid can be concentrated at least 5-fold.

In some non-limiting examples, a process can enrich the concentration of a species in a solution.

In some non-limiting examples, a process does not rely on manually removing a membrane for downstream analysis.

In some non-limiting examples, a process can enrich the concentration of a molecular species for downstream analysis.

In some non-limiting examples, a process can enrich the analyte of interest.

In some non-limiting examples, a device can directly remove a concentrated sample.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The following drawings are provided to help illustrate various features of non-limiting examples of the disclosure, and are not intended to limit the scope of the disclosure or exclude alternative implementations.

FIG. 1 shows a schematic illustration of a collecting system.

FIG. 2 shows a schematic illustration of a probe system.

FIG. 3 shows a schematic illustration of a probe, an elution chamber, and an elution device.

FIG. 4 shows a schematic illustration of the probe, the elution chamber, and the elution device of FIG. 3 in a different configuration to the configuration of FIG. 3.

FIG. 5 shows an example of an elution device and a probe.

FIG. 6 shows the probe being electrically connected to ground.

FIG. 7 shows a schematic illustration of the probe with the viruses coupled thereto placed into contact with a liquid solvent contained within an elution chamber.

FIG. 8 shows a probe being placed into contact with the liquid solvent.

FIG. 9 shows a schematic illustration of a heating system interacting with a probe, and a container defining an elution chamber that contains a liquid solvent.

FIG. 10 shows a schematic illustration of a heating system that interacts with a probe, and a container defining an elution chamber that contains a liquid solvent.

FIG. 11 shows a schematic illustration of another collecting system.

FIG. 12 shows a schematic illustration of another collecting system.

FIG. 13 shows a schematic illustration of another collecting system.

FIG. 14 shows a schematic illustration of a detection system.

FIG. 15 shows a schematic illustration of an analysis system.

FIG. 16 shows a schematic illustration of another analysis system.

FIGS. 17A and 17B show a flowchart of a process for collecting and analyzing particles.

FIG. 17C shows a flowchart of a process for collecting and analyzing particles.

FIG. 18 shows a general purpose (left) continuous monitoring of indoor environment's aerosol for detection of airborne virus. A mobile platform performs walk in the indoor environment of interest, collects aerosol from various locations using electrostatic precipitation, concentrates aerosol in water using vortex mixer, extracts viral nucleic acid from aerosol by heating the sample at 90° C. for 5 minutes, and then performs chemical detection of viral nucleic acid using RT-LAMP with virus-specific primers at 65° C. for 25 minutes (right).

FIG. 19 shows a process for on-site detection of virus in aerosol. The system specifically focuses on SARS-CoV-2, but the same process can be used for any other viruses (other microorganisms, etc.) by replacing the RT-LAMP primers.

FIG. 20 shows a containment cube for sampling the environment (left) with the containment cube having an air scrubber attached to purge FluoSpheres, and a schematic of the inside of the containment cube (right) where FluoSpheres were injected using nebulizers and a probe collector in the center.

FIG. 21 shows a graph from a titration experiment. The number of FluoSpheres were varied and were introduced into the nebulizers. The number of beads collected was measured in water using a flow cytometer. The ratio is roughly equal to 0.01.

FIG. 22 shows various graphs for an on-field condition experiment. For example, FIG. 22A shows a graph of the estimated concentration of beads injected in the tent and nebulization time (ti) for each of the experiments (i=1, 2, 3, 4). Dashed lines delimit the typical range of influenza virus concentration found in airplanes and day care centers during flu season. FIG. 22B shows the number of beads collected in water using the process described herein and a de-ionized water volume of 250 μL. The dashed line denotes the number of beads required (in a 250 μL solution) to reach the calculated RT-LAMP LoD. FIG. 22C shows the ratio between the number of beads collected in water and the estimated number of beads injected in the tent (constant). FIG. 22D shows an application scenario emulated by the four experiments.

FIG. 23 shows a graph of the concentration of particles collected. The concentration of the particles collected can be increased by reducing the extraction volume of de-ionized water. In the graph the solid line represents the ideal behavior (e.g., a reduction of volume does not affect the mechanics of the removal process) of the collected particles concentration with respect to the extraction volume. The red dots represent the four experiments conducted, each one with a different extraction volume (i.e., 50 μL, 100 μL, 150 μL, 250μL).

FIG. 24 shows graphs characterizing the RT-LAMP reactions. For example, FIG. 24A shows the amplification curves, which demonstrate that it takes a shorter time to amplify RNA with a higher amount of starting copies. NTC is a non-template control. The amplification time of 50-copy RNA is 14.4±2.1 minute (mean±SD, N=50). FIG. 24B shows a positive identification percentage of N=50 biologically independent experiments by the real-time PCR machine Roche LightCycler 480. The working volume per reaction is 25 μL.

FIG. 25 shows a schematic illustration of a processes and related devices for electrostatic repulsion of viral nucleic acids from a conductive collecting surface (A-C) and charge driven electrical migration and concentration of nucleic acids (D-H) in the context of an integrated viral detection device.

FIG. 26 shows a schematic illustration of a process along with implementing devices for detachment/enrichment of collected viral particles, nucleic acid extraction, and transfer in the chemical assay chamber. The device takes as its input the probe loaded with aerosol sampled from the air. The components of the device are: A) A miniaturized vortex mixer holding a tube into which the probe is transferred after sampling; B) A heated section of tubing which is regulated to be 90° C.; C) A fluid transfer system including a peristaltic pump is capable of transferring precise volumes of fluid through the device; D) An RT-LAMP reaction chamber, which is held at 65° C. via a heated shell and monitored for fluorescence by the readout system composed of the light source and detector.

FIG. 27 shows a prototype that was built to demonstrate a proof-of-principle for RNA enrichment via electrophoresis against a semipermeable membrane.

FIG. 28 shows a graph of the fold change of RNA content after membrane-based electrophoretic RNA enrichment in the prototype device.

FIG. 29 shows a schematic illustration of multiple different collecting systems. For example, the stand-alone collecting system can be portable and moveable, the portable AC plug-in function can also be portable and can be integrated within a cooling system or a portable air purifier, and the HVAC integrated function system can be integrated within an HVAC system.

FIG. 30 shows a flowchart of a process to react to detecting pathogens in the air (e.g., viruses).

FIG. 31 shows a flowchart of a process for detecting pathogens in the air (e.g., viruses).

FIG. 32 shows a ESP sampler with the characterized performance.

FIG. 33 shows a detachment device and an enrichment device.

### DETAILED DESCRIPTION OF THE PRESENT DISCLOSURE

As described above, current testing methods are largely inadequate for large settings. In other words, it can be increasingly difficult to chemically test each individual that travels within an enclosed space. The alternative is to undesirably prevent individuals from using the enclosed space at all. Some non-limiting examples of the disclosure provide advantages to these issues (and others) by providing improved systems and methods for detecting microorganism or viral loaded aerosols. For example, some non-limiting examples of the disclosure provide a collecting system that can collect aerosolized pathogens from the environment, can detect the presence (or concentration) of these pathogens within an enclosed volume, and can alert, notify, etc., an user, or implement one or more remedial actions to mitigate the transmission of the pathogens to other individual within the enclosed space. In some cases, the collecting system can even determine that a pathogen is aerosolizable, in which the pathogen has previously been unknown to be able to aerosolize. In some configurations, by repeatedly testing for airborne pathogens, the collecting system can minimize energy intensive remedial measures. For example, during the SARS-CoV-2 pandemic, in order to minimize transmission of airborne viral particles, HVAC systems were required to significantly increase the air flow within an enclosed space, even if the space was large enough to, with the current HVAC practices, not readily transmit airborne viral particles. Thus, a collecting system that can determine when or when not to increase the flow rate of air within an enclosed space to mitigate viral transmission can be desirable to improve energy efficiency of HVAC systems.

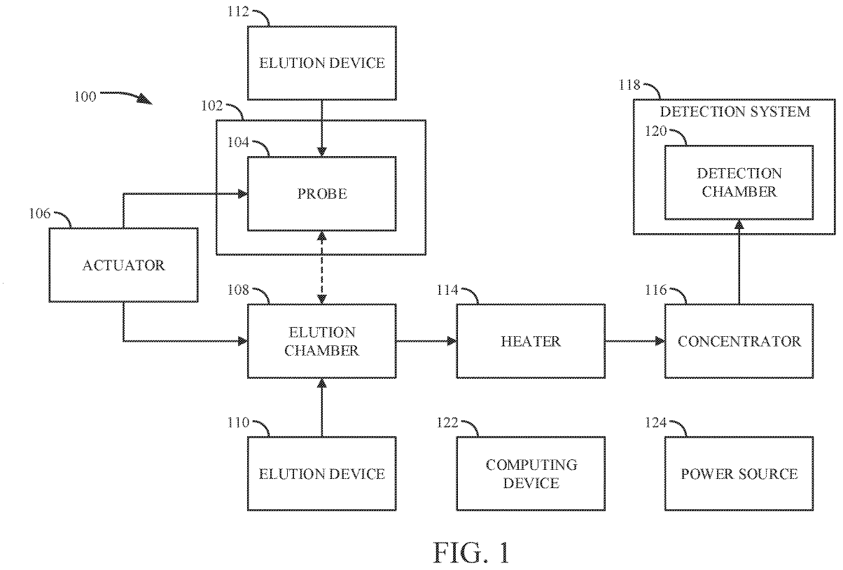

FIG. 1 shows a schematic illustration of a collecting system 100. The collecting system 100 can include a probe system 102, an actuator 106, an elution chamber 108, elution devices 110, 112, a heater 114, a concentrator 116, a detection system 118, a computing device 122, and a power source 124. The probe system 102 can include a probe 104 that can be configured to collect pathogens from a surrounding fluid. For example, the probe 104 can be in fluid communication with an enclosed volume (e.g., a room, a duct, vehicle, etc.) that includes aerosolized pathogens circulating therethrough. Periodically, aerosolized pathogens can contact the surface of the probe 104 and can remain on the probe 104, until, for example, liquid solvent elutes the collected pathogens into the liquid solvent. In some cases, the probe 104, which can be formed out of an electrically conductive material (e.g., a metal), can be electrically grounded (e.g., the probe 104 being electrically connected to an electrical ground). In this way, aerosolized pathogens (or droplets containing pathogens) that are ionized (e.g., with an ionizer) are electrically attracted to the electrically grounded probe 104. When these pathogens (or droplets) contact the probe 104, they lose their charge through the grounded probe 104, but remain affixed to the surface of the probe 104. In some cases, and as described below, the probe system 102 can include one or more ionizers, which can negatively (or positively) ionize particles in the surrounding fluid, which then attract and collect on the probe 104.

As shown in FIG. 1, the actuator 106 can be coupled to the probe 104, or can be coupled to the elution chamber 108. Regardless, the actuator 106 can be extended to position the probe 104 in the elution chamber 108, and can be retracted to remove the probe 104 from the elution chamber 108. Thus, the probe 104 can be in selective fluid communication with the elution chamber 108, via, for example, the actuator 106. The actuator 106 can be implemented in different ways. For example, the actuator 106 can be an electrical actuator (e.g., a linear actuator), a pneumatic actuator, a hydraulic actuator, etc., and can be electrically connected to the power source 124 (e.g., so that the power source 124 powers the actuator 106). In some cases, while not shown in FIG. 1, the collecting system 100 can include a robotic arm that can selectively grasp the probe 104 or the elution chamber 108 to place the probe 104 into (or out of) the elution chamber 108. Thus, the robotic arm can replace the actuator 106.

As described above, the probe 104 can be selectively placed into or out of the elution chamber 108. Thus, when, for example, the elution chamber 108 includes a liquid solvent (e.g., water, such as distilled water), the liquid contained in the elution chamber 108 can be selectively brought into (or out of) contact with a surface of the probe 104. The elution chamber 108 can be implemented in different ways. For example, the elution chamber 108 can be part of a container, such as, for example, a vial, a flask, etc., or can be part of a substrate in which fluid flows throughout the system. In this case, for example, the elution chamber 108 can be directed into the substrate, with a number of flow paths being in fluid communication with the elution chamber 108. In some non-limiting examples, the elution chamber 108 can have an internal volume less than 250 μL.

The elution devices 110, 112 can be configured to elute pathogens collected on the probe 104 into the elution chamber 108 (e.g., into the liquid solvent contained in the elution chamber 108), and each of the elution devices 110, 112 can be implemented in different ways. For example, each of the elution devices 110, 112 can be a mixer that mixes the liquid solvent contained in the elution chamber 108 to force the liquid solvent into contact with the probe 104 to elute the pathogens off the probe 104 and into the liquid solvent contained in the elution chamber 108. In some cases, the mixer can include a motor that is coupled to the elution chamber 108 to move the elution chamber 108 (e.g., relative to the probe 104), or coupled to the probe 104 to move the probe 104 (e.g., relative to the elution chamber 108). Thus, regardless, the mixer can cause relative movement between the probe 104 and the elution chamber 108 to agitate the liquid solvent contained in the elution chamber 108 so that the liquid solvent repeatedly comes into contact with the surface of the probe 104 to elute the pathogens of the probe 104 and into the liquid solvent. As another example, each of the elution devices 110, 112 can include an electrical conductor (e.g., an electrode) that can store a charge (e.g., a negative charge or a positive charge), and the electrical conductor can be placed into fluid communication with the liquid solvent contained in the elution chamber 108. In this case, the power source 124 can selectively charge the electrical conductor, which can draw pathogens or the genetic material of the pathogens away from the surface of the probe 104. For example, the electrical conductor that is negatively charged can be placed into close proximity to the surface of the probe 104, which can repel negatively charged species away from the electrical conductor (e.g., the pathogens that are themselves negatively charged, such as their outer membranes, or the genetic material of the pathogens that become negatively charged in an aqueous environment). Corresponding, including when the electrical conductor is positively charged, the electrical conductor can be placed into the liquid solvent away from the probe 104 and the negatively charged species can be attracted towards the electrical conductor and away from the surface of the probe 104. As yet another example, the elution device 110, 112 can be a power source (e.g., similar to the power source 124, an electrical charge generator, an electrostatic generator, a capacitor, an electrode, etc.) that can be in selective electrical communication with the probe 104. In this case, the power source can charge the probe 104 to repel the pathogens (or the genetic material of the pathogens) away from the surface of the probe 104.

In some non-limiting examples, after the pathogens have been eluted off the probe 104 into the liquid solvent of the elution chamber 108, the liquid containing the pathogens can flow through the heater 114 (or a component heated by the heater 114). For example, the heater 114 can heat the liquid containing the pathogens as the liquid flows through the collecting system 100 (e.g., to the concentrator 116 and the detection system 118). In some cases, this can include the heater 114 heating a component (e.g., a heating block) that defines a channel in which the liquid flows through (e.g., the channel can be directed into the substrate described previously with regard to the elution chamber 108). In other configurations, including prior to the pathogens being eluted off the probe 104, the heater 114 can also heat the elution chamber 108 itself, or the liquid solvent before, during, after, etc., being deposited into the elution chamber 108. In some non-limiting examples, the heater 114 can be configured to heat the liquid to a temperature for a period of time (and remain at or above the temperature for the period of time). In some cases, the temperature can be greater than 80° C., greater than 85° C., greater than 90° C., greater than 92° C., etc. In some cases, the temperature can be substantially (i.e., deviating by less than 10 percent from) 80° C., substantially 85° C., substantially 90° C., substantially 92° C., etc. In some configurations, the period of time can be less than 5 minutes, less than 15 minutes, less than 30 minutes, less than 45 minutes, less than 60 minutes, less than 75 minutes, etc.

Regardless of the configuration, the heater 114 can be configured to lyse the pathogens (e.g., that are contained in the liquid) to release the genetic material within each pathogen into the liquid. In some cases, the lysing (or in other words rupturing of the membrane, capsid, etc., that encapsulates the genetic material) occurs when a sufficient amount of heat has been added to the liquid and the pathogens. Thus, the temperature that is liquid is heated to, and the total time the liquid is heated for during the heating process can facilitate lysing of the pathogen to release the genetic contents. In other words, higher temperatures can require less time to lyse the pathogens. However, there is a point at which higher temperatures (or longer heat times) can undesirably degrade the genetic material. In some cases, then, heating the liquid to a temperature within a range between 65° C. and 92° C. can lyse the pathogens without damning the genetic contents within a reasonable amount of time (e.g., temperatures greater than 92° C. could degrade some genetic contents, such as, for example, single stranded ribonucleic acid (“RNA”)).

In some embodiments, the probe 104 can be placed into the elution chamber 108 and the liquid solvent within the elution chamber 108 can be heated while the probe 104 is placed therein. In this way, the pathogens captured on the probe 104 advantageously lyse their genetic contents within the liquid solvent regardless of whether or not the pathogens have decoupled from the surface of the probe 104 and have dispersed within the liquid solvent. Thus, even pathogens that still remain coupled to the surface of the probe 104 can still purge their genetic contents into the liquid solvent without having to be decoupled from the probe 104. Accordingly, this process can lead to a more accurate detection of the pathogens, since little to no genetic material is prevented from entering the liquid solvent. In some cases, the heater 114 can heat the liquid before or while the probe 104 is placed into the elution chamber 108, via a component (e.g., a heat block) in thermal communication with the elution chamber 108 and the liquid therein. In other cases, the heater 114 can directly heat the probe 104 thereby heating an exterior surface of the probe 104.

In some cases, including after the heater 114 has lysed the pathogens, the liquid that includes the genetic material can flow to the concentrator 116, which can concentrate the genetic material prior to directing the concentrated genetic material to the detection system 118. In some cases, the concentrator 116 can concentrate the genetic material 1 fold, 2 fold, 3 fold, 4 fold, 5 fold, etc. In some cases, the concentrator 116 can concentrate the genetic material at least 1 fold, at least two fold, at least three fold, at least four fold, at least 5 fold, etc. In some configurations, the concentrator 116 can include a concentration chamber, a semi-permeable membrane positioned in the concentration chamber, and at least one electrode. The at least one electrode can be charged to attract (or repel) the negatively charged genetic material towards the semi-permeable membrane. In some cases, because the semi-permeable membrane blocks passage of genetic material therethrough, but allows passage of liquids through (e.g., water), the genetic material concentrates within a portion of the concentration chamber. In some cases, concentrating the genetic material can significantly increase the limit of detection of the detection system 118. In this way, the probe collecting system 100 can advantageously sense even lower concentrations of pathogens in the surrounding fluid (e.g., lower concentrations of airborne pathogens in a given volume).

In some non-limiting examples, including after the concentrator 116 has concentrated the genetic material into concentrated genetic material, the concentrated genetic material (or the genetic material) and liquid can be directed to the detection system 118. For example, the genetic material (concentrated genetic material) can be directed to a detection chamber 120 of the detection system 118. In some cases, the detection chamber 120 can also function as a reaction chamber, however, in other configurations, the reaction chamber can be separate from the detection chamber 120. In this case, the reaction chamber can be positioned upstream of the detection chamber (or the genetic material can be placed into the reaction chamber before being placed into the detection chamber). Regardless, the genetic material (concentrated genetic material) can be multiplied (or in other words amplified) in the detection chamber 120 (or the reaction chamber). For example, the genetic material (e.g., when the genetic material is nucleic acid, such as single stranded RNA) can be subjected to a reverse transcriptase loop mediated isothermal amplification (“RT-LAMP”) reaction. Thus, reagents for the RT-LAMP can be introduced into the detection chamber (or the reaction chamber), such as, for example, one or more primers specific to a corresponding region on the genetic material (e.g., of a pathogen to be detected), a reverse transcriptase, a deoxyribonucleic acid (“DNA”) polymerase, etc. In addition, the heater 114 (or another heater) can also heat the detection chamber 120 (or the reaction chamber) to facilitate the amplification of the genetic material. For example, in some cases, the detection chamber 120 (or the reaction chamber) including the liquid contained therein can be heated to a temperature (e.g., less than or equal to 65° C., less than 98° C., less than 94° C.) for a period of time (e.g., greater than 15 minutes, less than 75 minutes, etc.) to facilitate the amplification reaction.

In some cases, including while the amplification reaction is occurring (e.g., in the detection chamber 120) the detection system 118 can monitor the number of copies of the genetic material. For example, a chemical substrate (e.g., a dye) can be introduced into the detection chamber 120, which fluoresces when the chemical substate interacts with the copied genetic material (e.g., the copied genetic material being double strand DNA (“dsDNA”)), but does not (or to a much smaller extent) when interacting with just the genetic material that is single stranded. As another example, the chemical substrate can be a probe primer that is cleaved during an amplification cycle (e.g., the probe primer is cleaved when one strand of genetic material is multiplied). When the probe is cleaved, the probe emits a fluorescent signal (e.g., from the fluorophore), but does not when the probe is not cleaved thereby leading to an amplification dependent increase in fluorescence. Regardless of the chemical substrate, the detection system 118 can monitor changes in the fluorescence signal during the amplification reaction, which can include generating a amplification curve (e.g., that is the fluorescence signal over time, with the fluorescence signal being dependent on the number of copies of the genetic material). Then, the collecting system 100 (e.g., the computing device 122 of the collecting system 100) can determine the initial amount of the (concentrated) genetic material using the amplification curve, and determine the concentration of the pathogens in the surrounding fluid (e.g., contained by the enclosed volume), based on the initial amount of the genetic material (and based on the total volume of the enclosed volume that includes the surrounding fluid, and an efficiency ratio of the collection rate of the pathogens in the surrounding volume to the pathogens collected on the probe).

In some non-limiting examples, after the analysis has been completed by the detection system 118, the collecting system can transmit (e.g., via the computing device 122) the results of the monitoring (e.g., including the amplification curve, the initial amount of genetic material, the concentration of the pathogens in the surrounding fluid, etc.) to another computing device (e.g., a smartphone, a server, etc.) to, for example, notify a user. In some cases, and as described below, the results of the monitoring (e.g., if the concentration of the pathogens in the surrounding fluid exceeds a threshold value), the collecting system 100 can implement one or more remedial actions, such as, for example, increase the flow rate of fluid in the surrounding fluid (e.g., by activating a fan), activate a disinfection system (e.g., turn on or increase the power of ultraviolet lights optically coupled to the surrounding fluid), etc.

In some configurations, utilizing a LAMP process (e.g., RT-LAMP) advantageously does not require a thermocycler, which can be bulky, non-portable, and can considerably increase the required detection time. For example, during a PCR process, a thermocycler heats and cools the genetic material during each cycle. In particular, a thermocycler can heat the solution to a first temperature that splits the strands of DNA to denature the DNA, cooling the solution to a second temperature that anneals the primers on the split DNA, and heating to the solution a third temperature that extends to copy the DNA (e.g., with the first, second, and third temperatures being different), which can define a cycle. This process can be repeated for multiple cycles. While a PCR reaction scheme may be more sensitive (e.g., increasing a collecting system limit of detection), the requirement to continually cycle the temperature can increase the detection time. Thus, LAMP (e.g., RT-LAMP) can provide a detection quicker than PCR.

In some configurations, the collecting system 100 can include a thermocycler (e.g., as being part of the heater 114, a combination of the heater 114 and the computing device 122, etc.). Thus, the collecting system 100 can be configured to implement a polymerase chain reaction (“PCR”) on the genetic material that is derived from pathogens collected by the probe 104. In this case, the thermocycler can implement one or more cycles on the genetic material to amplify the genetic material. For example, during one cycle, the thermocycler can increase the temperature of the genetic material to a first temperature that denatures the genetic material (e.g., greater than or equal to 95° C.), cool the genetic material to a second temperature that anneals one or more primers to the genetic material (e.g., less than or equal to 65° C.), and increase the genetic material to a third temperature that extends and copies the genetic material. Thus, the thermocycler can during one cycle, cycle between at least two different temperatures (e.g., three different temperatures), with each of the at least two different temperatures being within a temperature range that is between 60° C. and 95° C. In some cases, the PCR is a quantitative PCR (“qPCR”), in some configurations, the PCR can be reverse transcriptase qPCR (“RT-qPCR”). In some embodiments, the thermocycler can be portable (e.g., the power source 124 can power the thermocycler, in which case the power source 124 can be an electrical storage device such as a battery). In this case, the thermocycler can be advantageously used to detect the pathogens without being forced to be used in a laboratory setting.

In some embodiments, the thermocycler can be configured to implement a reverse transcriptase quantitative polymerase chain reaction (“RT-qPCR”) on the genetic material, or can be configured to implement a fast RT-qPCR on the genetic material. In some cases, for the RT-qPCR, the one or more cycles (e.g., with each cycle doubling the amount of genetic material after completion of the cycle), the one or more cycles (e.g., 40 cycles) can be completed in less than or equal to 2 hours. In some embodiments, for the fast RT-qPCR, the one or more cycles (e.g., 40 cycles) can be completed in less than or equal to 3 minutes, substantially 3 minutes, etc. In the fast RT-qPCR case, the thermocycler can be configured to rapidly cool and heat the genetic material (and the liquid solvent) to facilitate the relatively speedy result. In addition, the one or more reagents added to the genetic material (or preloaded), can be engineered to facilitate the faster result. For example, the reverse transcriptase for the fast RT-qPCR can be different (e.g., engineered differently) to be faster than the reverse transcriptase for the RT-qPCR As another example, the DNA polymerase for the fast RT-qPCR can also be different (e.g., engineered differently) to be faster than the reverse transcriptase for the RT-qPCR. In some configurations, the one or more cycles can be greater than 5 cycles, greater than 10 cycles, greater than 15 cycles, greater than 20 cycles, greater than 25 cycles, greater than 30 cycles, greater than 35 cycles, greater than 40 cycles, etc.

In some non-limiting examples, the computing device 122 can be in communication (e.g., bidirectional communication) with some (or all) of the components of the collecting system 100, as appropriate. For example, the computing device 122 can be in communication with the probe system 102, the actuator 106, the elution devices 112, 110, the heater 114, the concentrator 116, the detection system 118, etc. In addition, the computing device 122 can implement some (or all) processes described herein as appropriate, such as, for example, causing the actuator to move the probe 104 (or the elution chamber 108), causing the heater 114 to heat liquid that includes the pathogens, etc.

The computing device 122 can be implemented in a variety of ways. For example, the computing device 122 can be implemented as one or more processor devices of known types (e.g., microcontrollers, field-programmable gate arrays, programmable logic controllers, logic gates, etc.), including as general or special purpose computers. In addition, the computing device 122 can also include other computing components, such as memory, inputs, other output devices, etc. (not shown). In this regard, the computing device 122 can be configured to implement some or all of the steps of the processes described herein, as appropriate, which can be retrieved from memory. In some non-limiting examples, the computing device 122 can include multiple control devices (or modules) that can be integrated into a single component or arranged as multiple separate components.

The power source 124 can provide power to some (or all) of the components of the collecting system 100, as appropriate. For example, the power source can power the actuator 106, the probe system 102, the elution devices 112, 114, the heater 114, the concentrator 116, the detection system 118, the computing device 122, etc. In some cases, the power source 124 can include an electrical power source, such as, for example, an electrical storage device (e.g., one or more batteries, a capacitor such as a super capacitor, etc.).

In some non-limiting examples, although not shown in FIG. 1, the collecting system 100 can include one or more pumps that drive the liquid (e.g., the liquid solvent) through the collecting system 100. For example, the pump can direct fluid from the elution chamber 108, through the heater 114 (or a component heated by the heater), through the concentrator 116, and to the detection chamber 120. In addition, as described above, components of the probe collection system 100 can be integrated within a single substate, or can be other separate components. In this regard, for example, the elution chamber 108 can be removably coupled to the flow path that is configured to heat the liquid containing the pathogens, or other components, such as, for example, the concentrator 116, the detection chamber 120, etc.

While pathogens (e.g., microorganisms including bacteria, viruses, etc.) have been described mainly with regard to FIG. 1, in other configurations, the collecting system 100 can be defined as a collecting system 100 in which the collecting system 100 can collect (and detect) other analytes that are in the surrounding fluid. For example, these other analytes can include other microorganisms (e.g., those that are not virulent towards a particular animal species such as humans), other airborne particulates, etc. In some cases, the collecting system 100 can collect particles that are bacterial, mold, etc., spores. Then, a chemical can be added to the liquid solvent (or the liquid solvent can be preloaded in the elution chamber 108), which can lyse the spores (e.g., a bacterial spore, a mold spore, etc.) so that the spores release their genetic contents within the liquid solvent (e.g., the analyte).

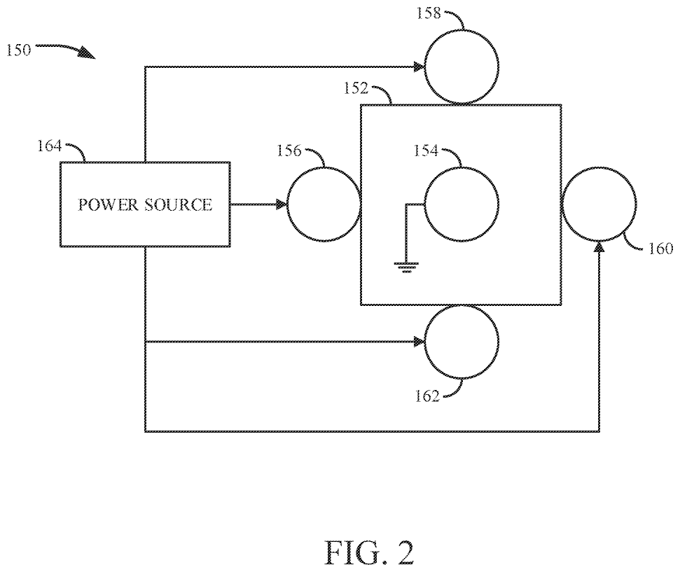

FIG. 2 shows a schematic illustration of a probe system 150, which can be a specific implementation of the probe system 102. Thus, the probe system 102 pertains to the probe system 150 (and vice versa). The probe system 150 can include a housing 152, a probe 154, ionizers 156, 158, 160, 162, and a power source 164. As shown in FIG. 2, the probe 154, and the ionizers 156, 158, 160, 162 can be coupled to the housing 152. In addition, the probe 154 can be removably coupled from the housing 152 (e.g., to facilitate easier removal of the collected pathogens). In some non-limiting examples, each ionizer 156, 158, 160, 162 can be electrically connected to the power source 164, and the power source 164 can charge (e.g., negatively charge) each ionizer 156, 158, 160, 162 to ionize particles including airborne pathogens pass by an ionizer. In some cases, each ionizer 156, 158, 160, 162 can include a capacitor that is configured to be charged (e.g., negatively charged) at a voltage. In some cases, this voltage can be relatively high, such as being, for example, greater than or equal to 20 kV. Regardless, each ionizer 156, 158, 160, 162 can be configured to create a corona discharge, which can charge the fluid surrounding each ionizer 156, 158, 160, 162. In this way, each ionizer 156, 158, 160, 162 charges the surrounding fluid (e.g., the air surrounding an ionizer) thereby charging the particles (e.g., the airborne pathogens) in the surrounding fluid. In some cases, the probe 154 can be electrically connected to an electrical ground. In this way, the charged particles (created by the ionizers 156, 158, 160, 162) attract to the probe 154 and are collected on the surface of the probe 154. In some cases, because the probe 154 is grounded, charge does not build up on the probe 154, so that the probe 154 can continually attract the charge particles in the surrounding fluid.

In some non-limiting examples, while the probe system 150 has been illustrated as having four ionizers 156, 158, 160, 162, in other configurations, the probe system 150 can have other numbers of ionizers (e.g., one, two, three, five, etc.). In addition, while the probe system 150 has been illustrated as having the probe 154, the probe system 150 can have other numbers of the probes (e.g., two, three, four, etc.) each of which can be structured in a similar manner to the probe 154 (e.g., the probe 154 being coupled to the housing 152) In some cases, having additional probes can be advantageous in that multiple probes can collect more airborne particles. As shown in FIG. 2, each of the ionizers 156, 158, 160, 162 are positioned relative to the probe 154 so that the ionizers 156, 158, 160, 162 surround the probe 154. For example, the probe 154 can be positioned between a first pair of ionizers (e.g., the ionizers 156, 160) and can be positioned between a second pair of ionizers (e.g., the ionizers 158, 162).

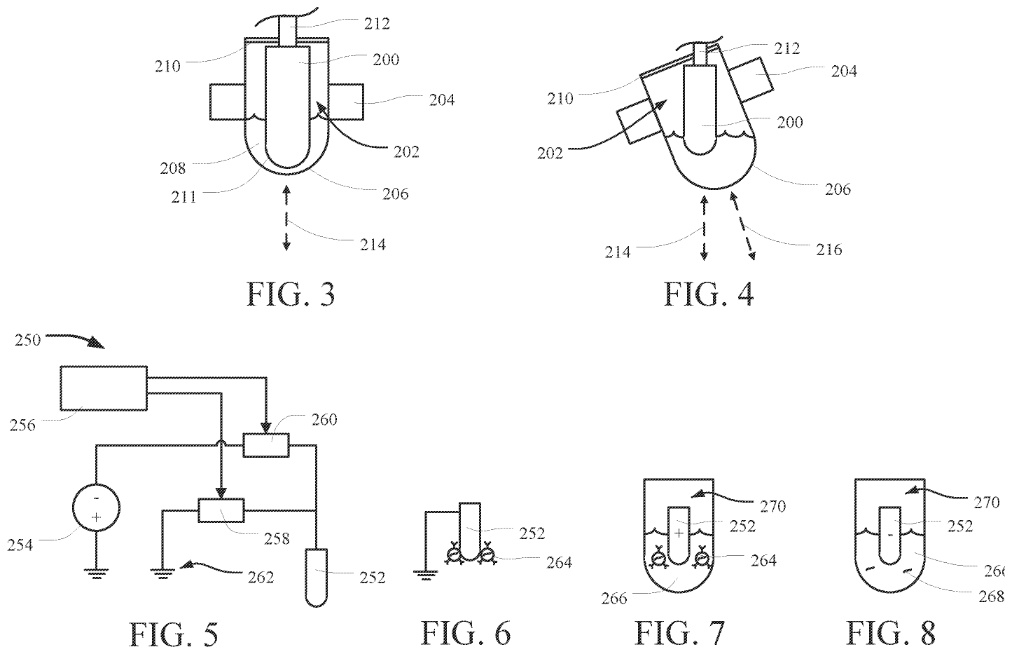

FIG. 3 shows a schematic illustration of a probe 200, an elution chamber 202, and an elution device 204, each of which can be a specific implementation of the probe 104, the elution chamber 108, and the elution devices 110, 112. Thus, these components pertain to each other. As shown in FIG. 3, a container 206 can define the elution chamber 202 (e.g., the internal volume of the container 206 being the elution chamber 202), which can contain a liquid solvent 208 (e.g., water, distilled water, deionized water, etc.). In some non-limiting examples, the container 206 (or the collecting system generally) can include a seal 210 that can extend partially (or entirely) around the a hole of the elution chamber 202. In some cases, the seal 210 can include a hole to facilitate insertion of the probe 200 into the elution chamber 202. The probe 200 can be coupled to an actuator (not shown, but similarly to the actuator 106), and in some cases, the probe 200 can be removably coupled to the extension 212 (e.g., so that the probe 200 can move within the elution chamber 202). As shown in FIG. 3, the seal 210, which can be a membrane (e.g., that is retractable), can isolate the elution chamber 202 (and the liquid solvent 208) from the ambient environment. Thus, the seal 210 can block movement of the liquid solvent 208 out of the elution chamber 202, during, for example, mixing of the liquid solvent 208 (e.g., to prevent undesirably losing solvent that can contain the pathogens).

In some non-limiting examples, the probe 200 can be inserted through the hole of the seal 210, and the seal 210 at the hole can retract around a portion of the probe 200 to seal an exterior surface 210 of the probe 200 within elution chamber 202. In some cases, this portion of the probe 200 can be an extension 212 that is coupled to an end of the probe 200 and extends away from the probe 200. In some cases, the extension 212 can have a smaller cross-section than a portion of the probe 200 that has the exterior surface 210 (e.g., to better seal the probe 200 within the elution chamber 202). As shown in FIG. 3, the elution device 204 can include a motor (e.g., an electrical motor) that engages with the container 206. In some cases, the motor can rotate around an axis 214, which can be parallel to a longitudinal axis of the elution chamber 202, a longitudinal axis of the probe 200, a longitudinal axis of the container 206, etc. The motor can be configured to rotate the container 206 (and thus the elution chamber 202 and the liquid solvent 208) around the axis 214 in a first rotational direction (and an opposite second rotational direction). In some cases, the motor can cause the liquid solvent 208 to form a vortex, which can better elute off particles collected on the probe 200. In some cases, when the longitudinal axis of the probe 200 and the longitudinal axis of the elution chamber 202 are parallel (e.g., substantially parallel), a vortex is more likely to form to better elute off the particles. In some cases, the motor can reverse the rotational direction about the axis 214 to introduce turbulence in the liquid solvent 208, which can more aggressively force the liquid solvent 208 into contact with the exterior surface 211 of the probe 200 to better elute the particles off the probe 200 and into the liquid solvent 208. Regardless of the configuration, movement of the liquid solvent 208 by the motor causes the liquid solvent 208 to repeatedly contact the exterior surface 211 of the probe 200 thereby eluting the particles collected by the probe 200, off the probe 200 and into the liquid solvent 208.

FIG. 4 shows a schematic illustration of the probe 200, the elution chamber 202, and the elution device 204 in a different configuration to the configuration of FIG. 3. For example, FIG. 4 shows the probe 200 being angled relative to the container 206. In particular, a longitudinal axis of the probe 200 is parallel to the axis 214, but the axis 216 in which the motor of the elution device 204 rotates about is not parallel to the axis 214. In other words, the axis 216 is angled relative to the axis 214 (e.g., with both axes 214, 216 being in the same plane). In some cases, the axis 216 can be parallel to a longitudinal axis of the container 206. In some cases, because the probe 200 is angled relative to the axis of rotation of the container 206, rotation of the chamber 202 by the motor can increase the agitation of the liquid solvent 208, which can facilitate better eluting of the particles off the probe 200 and into the liquid solvent 208 (e.g., the increased agitation causing the liquid solvent 208 to more forcefully contact the exterior surface 211 of the probe 200).

FIG. 5 shows an example of an elution device 250 and a probe 252, each of which can be specific implementations of the elution devices and the probes described herein. Thus, the other elution device and the probes described herein pertain to the elution device 250 and the probe 252 (and vice versa). The elution device 250 can include a power source 254, a computing device 256, and switches 258, 260. As shown in FIG. 5, the switch 258 can be electrically connected to an electrical ground 262 and the probe 252, while the switch 260 can be electrically connected to the power source 254 and the probe 252. In some cases, the switches 258, 260 can be implemented in different ways. For example, the switches 258, 260 can be electrical relays, transistor switches, etc. Regardless of the configuration, each switch 258, 260 can be controlled by the computing device 256, and each switch 258, 260 can switch between being opened and closed. In this way, the computing device 256 can selectively change the property of the probe 252. For example, the computing device 256 can cause the switch 260 to open (e.g., breaking the circuit), and can cause the switch 258 to close (e.g., closing the circuit). In this case, the probe 252 electrically connects to the electrical ground 262 thereby facilitating particulate collection on the probe 252. As another example, the computing device 256 can cause the switch 258 to open, and can cause the switch 260 to close. In this case, the power source 254 including a capacitor of the power source 254 electrically connects to the probe 252 to charge the probe 252 (e.g., which in this case includes negatively charging the probe 252) thereby repelling charged particles (e.g., negatively charged particles). Thus, the computing device 256 can select whether to ground or charge (e.g., negatively charge) the probe 252, based on the desired step of the process (e.g., grounding during collecting, and charging during analysis).

FIG. 6 shows the probe 252 being electrically connected to ground, which can facilitate the collection of viruses 264 on the surface of the probe 252. In some cases and as shown in FIG. 7, the probe 252 with the viruses 264 coupled thereto can be placed into contact with a liquid solvent 266 contained within an elution chamber 270. At this point, the viruses 264 disperse within the liquid solvent 266, and in some cases, the capsid of each of the viruses 264 can generate a positive charge when interacting with the liquid solvent 266 (e.g., solvating in the liquid solvent 266, which can be water). Then, including after a period of time, a computing device can cause the probe 252 to be positively charged (e.g., when the power source 254 has a positive voltage) by, for example, closing the switch 260 so that the power source 254 electrically connects to the probe 252. At this point, the positively charged viruses 264 are repelled away from the positively charged probe 252 to help facilitate eluting of the viruses 264 off or away from the surface of the probe 252.

FIG. 8 shows the probe 252 being placed into contact with the liquid solvent 266. However, the viruses 264 have been lysed (e.g., the capsids have been ruptured) to release genetic material 268 within the liquid solvent 266. In some cases, as described above, this can be completed by heating the liquid solvent 266 (and thus the viruses 264). As shown in FIG. 8, the probe 252 is negatively charged (e.g., by the computing device 256 closing the switch 260), which repels the genetic material 268 that is negatively charged (e.g., RNA becomes negatively charged in water) to facilitate eluting of the genetic material 268 off or away from the surface of the probe 252.

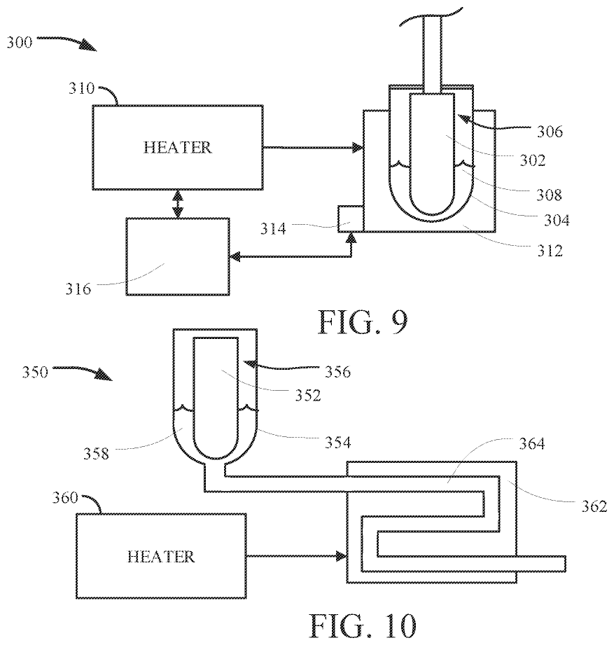

FIG. 9 shows a schematic illustration of a heating system 300 interacting with a probe 302, and a container 304 defining an elution chamber 306 that contains a liquid solvent 308. The heating system 300 can include a heater 310, a component 312 to be heated by the heater 310, a temperature sensor 314, and a computing device 316. As shown in FIG. 9, the component 312 can be a heating block that can have a recess that receives the container 304. In this way, the heating block can be in better contact with the container 304 to heat the container 304 and thus the liquid solvent 308. In some cases, the heating block can have other shapes other than a prism (e.g., a rectangular prism). In other cases, the component 312 can be the container 304, so that there is no intermediary heating component, and thus the heater 310 can directly heat the container 304 (e.g., through the air). In some non-limiting examples, the temperature sensor 314 can be in thermal communication with the component 312 (e.g., the temperature sensor 314 can be coupled to the component 312) to sense the temperature of the component 312. The computing device 316 can be in communication with the heater 310 and the temperature sensor 314. In this way, the computing device 316 can turn on the heater 310 to heat the component 312 when the temperature of the component 312 is below a threshold temperature (e.g., 90° C.), and the computing device 316 can turn off the heater 310 when the temperature of the component 312 is at (or above) the threshold temperature. Thus, the computing device 316 can ensure that the component 312 is at a consistent temperature (e.g., to facilitate lysing of the collected microorganisms or viruses).

In some non-limiting examples, the component 312 to be heated can be formed out of a material that is configured to retain heat. For example, the component 312 can be formed out of a material with a relatively high thermal conductivity, such as metals (e.g., aluminum, brass, steel, etc.). In some cases, the heater 310 can be an electrical heater that can be powered by a power source (e.g., the power source 124). In this case, the electrical heater can have one or more restive heating elements that receive electrical power to emit heat.

FIG. 10 shows a schematic illustration of a different heating system 350 that interacts with a probe 352, and a container 354 defining an elution chamber 356 that contains a liquid solvent 358. The heating system 350 can include a heater 360 and a component 362 to be heated by the heater 360. As shown in FIG. 10, the component 362 can be a heating block having a channel 364 that is directed through the heating block. In some cases, the channel 364 can have no turn, or one or more turns, which can facilitate better heat transfer between the component 362 and the liquid solvent 358 that flows through the channel 364. As shown in FIG. 10, the chamber 358 can be in fluid communication with the channel 364. For example, after the liquid solvent 358 elutes the pathogens off the probe 352, the chamber 358 can be brought into fluid communication with the channel 364 (e.g., with the chamber 358 not initially being in fluid communication with the channel 364). Then, the liquid solvent 358 can be driven (e.g., by a pump not shown in FIG. 10) from the elution chamber 356 and through (and out) the channel 364. As the liquid solvent 358 is loaded in and paused for a defined time, then driven through the channel 364, the pathogens in the liquid solvent 358 are lysed (e.g., by the increased temperature) and thus release their genetic material in the liquid solvent 358. In some cases, the length of the channel 364 can correspond to the time needed to lyse the pathogens and the flow rate of the liquid solvent 358 through the channel 364. In this way, the liquid solvent 358 is forced to be in thermal communication with the component 362 for at least the time necessary to lyse the pathogens in the liquid solvent 358. In other words, all of the liquid solvent 358 travels through the channel 364 for the time necessary to lyse the pathogens, so that when the liquid solvent 358 exits the channel 364, all the pathogens are lysed. In some cases, the total volume of the channel 364 is greater than the volume of the liquid solvent 358, and in some cases, the total volume of the channel 364 can be substantially the same as the total volume of the chamber 356. In some non-limiting examples, while not shown in FIG. 10, the heating system 350 can also include a temperature sensor and a computing device.

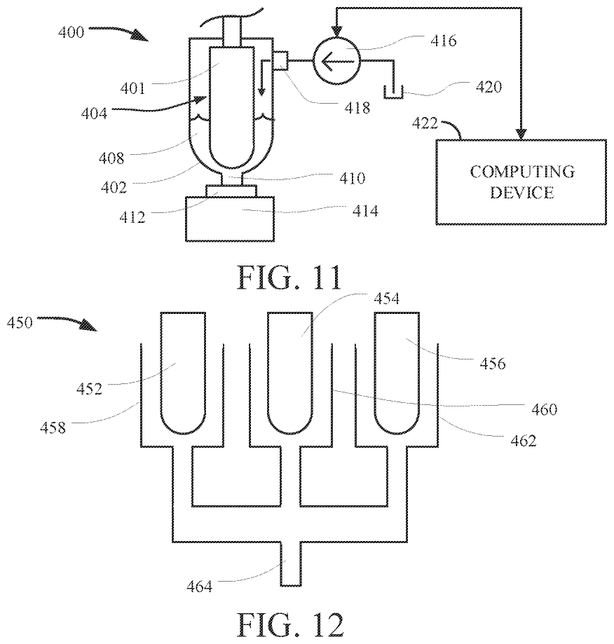

FIG. 11 shows a schematic illustration of a collecting system 400 that can include a probe 401, a container 402 defining an elution chamber 404 containing a liquid solvent 406, after, for example, analytes (e.g., pathogens) have been eluted off the probe 401. In some non-limiting examples, the container 402 can include a port 410 that can interface with a corresponding port 412 of a channel 414. In this way, after elution of the pathogens (e.g., by mixing the liquid solvent 406), the container 402 can be brought into fluid communication with the channel 414 by, for example, coupling the ports 410, 412 together. In other configurations, the container 402 can be in fluid communication with the channel 414 during the elution process (e.g., when an elution device elutes off analytes into the liquid solvent 408). In some configurations, the collecting system 400 can include a pump 416 can be in fluid communication with the elution chamber 404 (e.g., via a port 418 of the container 402) to drive fluid into the elution chamber 404 to drive the liquid solvent 406 into the channel 414. For example, liquid can be driven by the pump 416 from a reservoir 420 (that can also be included in the collecting system 400) and into the elution chamber 404 to drive the liquid solvent 408 and the analytes therein into the channel 414. In some cases, driving liquid towards the probe 401 to contact the probe 401 can be advantageous in that the liquid can further elute off particles (e.g., residual particles) left on the probe 401. In some non-limiting examples, the pump 416 can drive fluid into the elution chamber 404 when the elution device is activated. For example, when the probe 401 is charged, or when a conductor that is positioned away of the probe 401 is charged (e.g., an electrode), particles (e.g., pathogens) can be (temporality) drawn away from the surface of the probe 401. However, with the pump 416 forcing the liquid solvent 408 away from the probe, the particles can be trapped within the liquid solvent 408 (e.g., so that the particles do not reattach to the probe 401). In some non-limiting examples, the collecting system 400 can include a computing device 422 that can be in communication with the pump 416 to cause the pump 416 to pump fluid.

FIG. 12 shows a schematic illustration of a collecting system 450, which can be a specific implementation of the collecting system 100 (or the other collecting systems described herein). Thus, the collecting system 100 (and the others) pertains to the collecting system 450 (and vice versa). The collecting system 450 can include probes 452, 454, 456, elution chambers 458, 460, 462, and a channel 464. In some cases, the channel 464 can be in fluid communication (or selective fluid communication) with each of the elution chambers 458, 460, 462. As shown in FIG. 12, each probe 452, 454, 456 can be positioned within a respective elution chamber 458, 460, 462. In this way, the particles collected on each probe 452, 454, 456 can be eluted off into the respective elution chamber 458, 4560, 462, and can subsequently flow into the channel 464. Thus, the usage of multiple probes can increase the number of particles collected (e.g., analytes, pathogens, etc.).

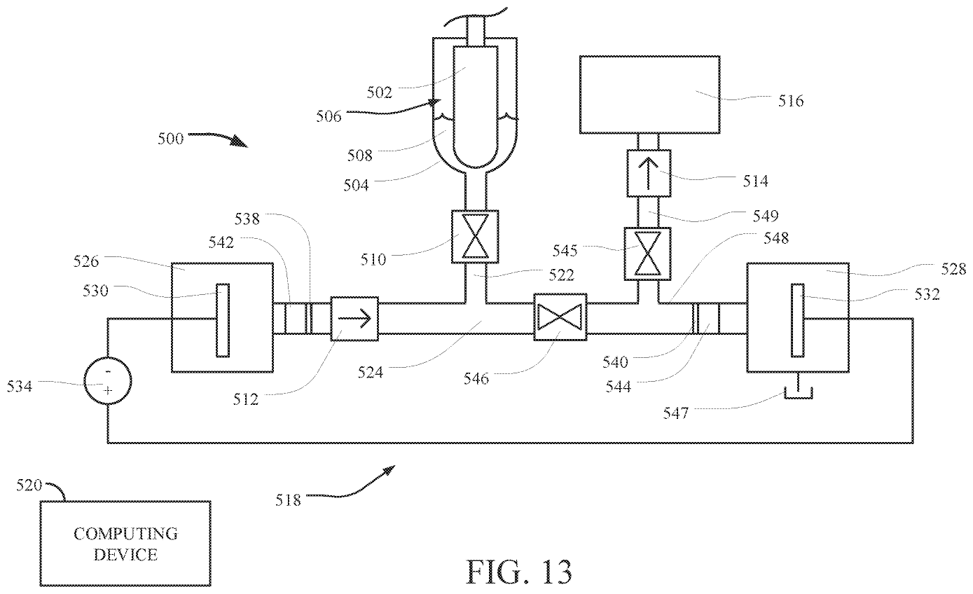

FIG. 13 shows a schematic illustration of a collecting system 500, which can be a specific implementation of the collecting system 100 (or the other collecting systems described herein). Thus, the collecting system 100 (and the others) pertains to the collecting system 500 (and vice versa). The collecting system 500 can include a probe 502, a container 504 defining an elution chamber 506 having liquid solvent 508 positioned therein, a valve 510, pumps 512, 514, a detection chamber 516, a concentrator 518, and a computing device 520. In some cases, the computing device 520 can be in communication with some or all of the components of the collecting system 500, as appropriate. For example, the computing device 520 can cause the valve 510 to open (and close), and can cause the pumps 512, 514 to draw fluid through the collecting system 500.

In some cases, including during elution of particles off the probe 502 (e.g., by an elution device), the valve 510 can be closed to isolate the elution chamber 506 from other components of the collecting system 500. However, in other cases, the valve 510 can open (e.g., by the computing device) and the pumps 512, 514 can draw fluid out of the elution chamber 506, through the valve 510, and into a channel 522 that is in fluid communication with the concentrator 518, during, for example, elution of the particles off the probe 502 by an elution device. In some cases, once the liquid solvent 508 has been directed into the channel 522 (or the concentrator 518), the computing device 520 can close the valve 510 (e.g., to prevent backflow of the liquid solvent 508 back into the elution chamber 506).

The concentrator 518 can include a channel 524 (e.g., that can be in fluid communication with the channel 522), chambers 526, 528, electrodes 530, 532, a power source 534, barriers 538, 540, substrates 542, 544, and a valve 546. As shown in FIG. 13, the electrode 530 can be positioned within the chamber 526, while the electrode 532 can be positioned within the chamber 528. In some cases, the chambers 526, 528 can be in fluid communication with the channel 524. In some configurations, the chambers 526, 528 can have a larger cross-section than the channel 524, which can facilitate larger electrodes 530, 532, thereby allowing for generating larger electric fields. In some configurations, the chambers 526, 528 are not sealed from the surrounding air (e.g., to allow for the escape of evolved gases). In other cases, the electrodes 530, 532 can be positioned within the channel 524. Each electrode 530, 532 can be electrically connected to the power source 534 to charge the electrodes 530, 532. For example, the power source 534 can be electrically connected to the electrode 530 to charge the electrode 530 (e.g., to negatively charge the electrode 530,), and the power source 534 can be electrically connected to the electrode 532 (e.g., to positively charge the electrode 532). In some cases, one of the electrodes 530, 532 can be grounded, rather than, for example, one of the electrodes 530, 532 being electrically connected to a power source. In this case, the electric field generated would be lower, but would prevent the need for an additional power source. In some embodiments, the concentrator 518 can include at least two power sources. For example, a first power source can be electrically connected to the electrode 530 and can provide a higher magnitude voltage to the electrode 530 than the magnitude of the voltage applied to the electrode 532 by a second power source. In this way, with the multiple power source, the voltage differential between the electrodes 530, 532 can be tailored to the desired electric field, base on, for example, the properties of the analyte to be concentrated (e.g., the voltage applied to the electrodes is tied less to a single power source).

As shown in FIG. 13, each of the barriers 538, 540, and each of the substrates 542, 544 can be positioned in the channel 524. Each of the barriers 538, 540, and the substrates 542, 544 can be semi-permeable. For example, each barrier 538, 540, and each substrate 542, 544 can be permeable to a liquids, gases, etc., such as, for example, the liquid solvent 508 that contains the analyte (e.g., the genetic material), but can be impermeable to analytes (e.g., the genetic material including nucleic acids, such as, DNA, RNA, etc.). In this way, the analyte (e.g., the genetic material) can be blocked from passing past the barrier 540 (or the barrier 538). In some cases, each of the barriers 538, 540 can be positioned between the substrates 542, 544. For example, the substrate 542 can be positioned behind the barrier 538 (e.g., upstream of the barrier 538), while the substrate 544 can be positioned in front of the barrier 540 (e.g., downstream of the barrier 540). The substrates 542, 544 can each contact a respective barrier 538, 540 and can each be configured to mitigate convection of the fluid flowing through the channel 524 (e.g., the liquid solvent 508). In some cases, each substrate 542, 544 can be formed out of a polymer (e.g., a hydrogel, agarose, etc.). In some non-limiting examples, each substrate 542, 544, and each barrier 538, 540 can extend entirely across the channel 524. In some non-limiting examples, each barrier 538, 540 can be a membrane.

In some embodiments, the barriers 538, 540 together, the substrates 542, 544 together, or one barrier and one substrate on opposing sides (e.g., the substate 542 and the membrane 540) can ensure that the liquid (e.g., that is electrically conductive) is prevented from escaping (e.g., into the ambient environment). In some configurations, each substate 542, 544 can be anticonvective. For example, each substrate 542, 544 can mitigate (or block) convective flow of liquid through the channel 524 (and between the chambers 526, 528 that include the electrodes), which can undesirably disrupt the concentration gradient that develops during the electrophoresis process. In some configurations, the channel 524 can be filled with (e.g., entirely filed with, such as a height dimension being entirely filled) a polymer liquid solution. For example, the polymer liquid solution (e.g., including agarose) can also mitigate or block convection.

As shown in FIG. 13, the valve 546 can be positioned between the barriers 538, 540, the substrates 542, 544, and the electrodes 530, 532. For example, the valve 546 can be positioned behind the barrier 540 (and the substrate 544), which can include the valve 546 being positioned upstream of the barrier 540 (and the substrate 544). In some cases, a section 548 of the channel 524 can be positioned between the barrier 540 and the valve 546. In this way, when the analyte (e.g., the genetic material) concentrates within the channel 524, the valve 546 can be closed (e.g., by the computing device 520) to block fluid communication between the section 548 of the channel 524 from other portions of the channel 524. In other words, closing of the valve 546 can advantageously trap the concentrated analyte within the section 548 of the channel 524 between the valve 546 and the barrier 540. In some cases, the section 548 of the channel 524 can have a smaller cross-section than other portions of the channel 524. In this way, because the volume of the section 548 of the channel 524 is smaller, the analyte can advantageously be further concentrated. In some cases, the cross-section of the section 548 of the channel 524 can decrease along a length of the section 548 of the channel 524 (e.g., along the flow direction of the section 548 of the channel 524). In this way, the electric field density along the section 548 of the channel 524 can advantageously increase, which can increase the concentration of the analyte within the section 548 of the channel 524 (e.g., the section 548 of the channel 524 can have electric field gradient focusing). In some configurations, the concentrator 518 can include a reservoir 547 that can be in fluid communication with each chamber 526, 528, the pump 512, and the channel 524. In this way, fluid (e.g., a liquid, including an electrically conductive liquid) can be drawn from the reservoir 547, directed through the channel 524, through the barrier 540, through the substrate 544, and back into the reservoir 547 (e.g., via the chamber 528).

As shown in FIG. 13, the concentrator 518 can include a channel 549 (e.g., that can be substantially linear) that is in fluid communication with the channel 524 (e.g., at the section 548 of the channel 524) and the detection chamber 516. In some cases, the concentrator 518 can include a valve 545 that can be positioned within the channel 549 and can block fluid communication between the detection chamber 516 and the other portions of the collecting system 500 including the channel 524, such as the section 548 of the channel 524 (e.g., when the valve 545 is closed). For example, the pump 514 (or the pump 514) can draw fluid from the section 548 of the channel 524, through the valve 545 (e.g., when the valve 545 is open), and into the detection chamber 516. Then, the valve 545 can be closed (e.g., via the computing device 520), and the fluid can be trapped within the detection chamber 516.

In some non-limiting examples, the probe 502 can collect particles that can be the analyte (e.g., pathogens), or can contain the analyte (e.g., encapsulating the analyte, such as when the entity is a pathogen and the analyte is the genetic material of the analyte), etc. As described above, the particles can be eluted off the probe 502 and into the liquid solvent 508 by an elution device. In some cases, while the elution device elutes of the particles off the probe 502, the pump 512 (or another pump, such as the pump 416), can drive fluid (e.g., liquid such as liquid that is the same type as the liquid solvent 508) into the elution chamber 506 to capture the particles and ensure that the particles do not reattach to the probe 502. In addition, while the elution device elutes the particles off the probe 502, the valve 510 can be closed (e.g., to ensure that a higher percentage of particles are eluted into the solution), or can be opened to ensure that the particles do not reattach to the probe 502, but which can use an additional amount of liquid solvent (e.g., which can undesirably decrease the concentration of the particles). Regardless of the configuration, after the particles have been eluted into the liquid solvent 508 to create a particle solution (e.g., that includes eluted particles and the liquid solvent 508), the particle solution can enter the concentrator 518. For example, the particle solution can be driven into the channel 524 (e.g., through the valve 510) by the pump 512 (or another pump), and the valve 510 can be closed, after the particle solution enters the channel 524 (e.g., to isolate the particle solution within the channel 524). Then, the concentrator 518 can concentrate the particle solution to form a concentrated particle solution (e.g., the concentrated particle solution having a higher concentration of the particles). For example, the particles can be charged, and the particles can be attracted to the electrode 532, while being repelled from the electrode 530. As a more specific examples, when the electrode 530 is negatively charged, when the electrode 532 is positively charged, and the particles are negatively charged (e.g., being genetic material), the particles can be drawn towards the electrode 532 and away from the electrode 530. As another specific example, when the electrode 530 is positively charged, the electrode 532 is negatively charged, and the particles are positively charged, the particles can be drawn towards the electrode 532 and away from the electrode 530. Regardless of the configuration, the particles are directed towards and congregate near the barrier 540 to concentrate near the barrier 540. In addition, fluid (e.g., liquid) can be directed towards the barrier 540 (e.g., by the pump 512 being a piston pump), which can force the particles close to (or against) the barrier 540, while the fluid passes through the barrier 540 (e.g., because the barrier 540 is semi-permeable). In this way, flow of fluid in a direction towards the barrier 540 can concentrate the analyte. In some configurations, the concentrator 518 can include an actuator (e.g., as part of a piston pump) that can be advanced to increase the pressure within the channel 524 (e.g., the actuator being advanced to contact the liquid). For example, the actuator can be in pressure communication with the channel 524 between the barriers 538, 540 (e.g., or upstream of the barrier 540), and as the actuator increases the pressure within the channel 524, the analytes that are blocked by the barrier 540 are forced to concentrate within the section 548 of the channel 524 (e.g., similarly to a reverse osmosis process). Regardless of the configuration, the concentrator 518 concentrates the particle solution at the section 548 of the channel 524 to form a concentrated particle solution.

In some non-limiting examples, the valve 545 can be closed while the concentrator 518 concentrates the particles. In this way, the particles do not move into the detection chamber 516 (e.g., via diffusion). In some cases, including after the concentrated particle solution has been formed, the valve 546 can be closed (e.g., by the computing device 520). In this way, the concentrated particle solution is advantageously trapped within the section 548 of the channel 524 so that, for example, the concentrated particle solution does not become diluted by diffusion. In addition, including after the valve 546 has been closed, the valve 545 can open, and the concentrated particle solution can be directed into the detection chamber 516 to detect the particles (e.g., by the pump 514 drawing the concentrated particle solution into the detection chamber 516). As described above, the detection chamber 516 can also be a reaction chamber, in that the particles can multiply within the detection chamber 516.

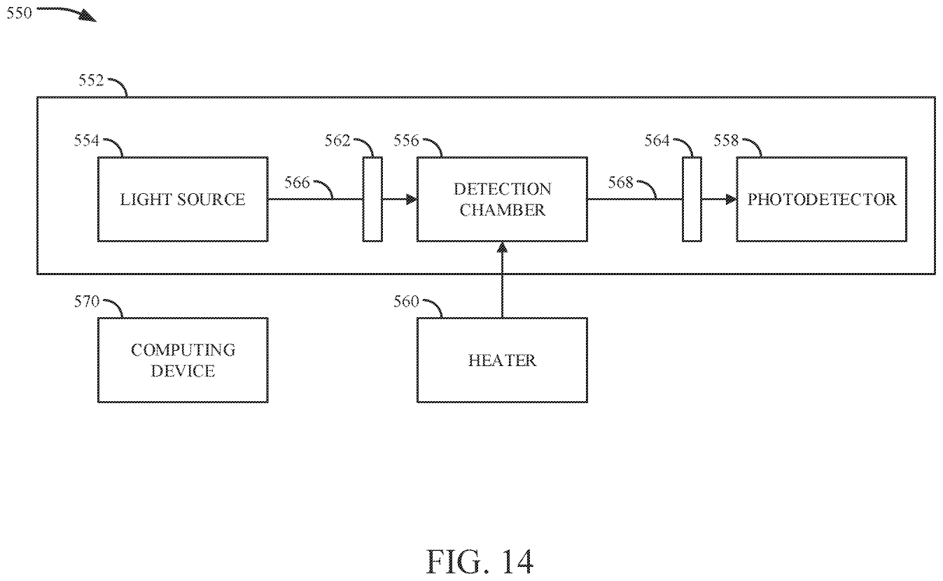

FIG. 14 shows a schematic illustration of a detection system 550, which can be a specific implementation of the detection system 118 (or the other detection systems described herein). Thus, the detection system 118 (and the others) pertains to the detection system 550 (and vice versa). The detection system 550 can include a chamber 552, a light source 554, a detection chamber 556, a photodetector 558, a heater 560, and optical filters 562, 564. As shown in FIG. 14, the light source 554, the detection chamber 556, and the photodetector 558, and the optical filters 562, 564 can each be positioned within the chamber 552. In this way, light from the ambient environment does not undesirably interact with the photodetector 558 (or the detection chamber 556). For example, the chamber 552 can be partially (or fully) enclosed from the ambient environment, and the internal volume of the chamber 552 can be isolated from energy entering the chamber 552 from the ambient environment. Thus, in some cases, the chamber 552 can be formed by one or more walls of a reflective material (e.g., a metal) that faces the ambient environment. In this way, light from the ambient environment that is directed at the chamber 552, reflects off the reflective material and propagates away from the chamber 552, rather than, entering into the chamber 552 and undesirably interacting with the photodetector 552.

In some non-limiting examples, the light source 554 can be configured to emit light 566 towards the detection chamber 556, which can interact with a substrate that interacts with the particles within the detection chamber 556. For example, the light 566 can excite the substrate within the detection chamber 556 (e.g., via fluorescence), and the substrate can emit light 568 towards the photodetector 558 that can be different than the light 566. For example, the light 568 can have a larger wavelength than the light 566. In some cases, the optical filters 562, 564 can each filter light to increase the signal to noise ratio. For example, the optical filter 562 can be optically coupled to the light source 554 (and the detection chamber 556) and can be positioned between the light source 554 and the detection chamber 556, while the optical filter 564 can be optically coupled to the photodetector 558 (and the detection chamber 556) and can be positioned between the detection chamber 556 and the photodetector 558. The optical filter 562 can block light from passing through the optical filter 562 (and to the photodetector 558) that has a wavelength that is the same as a wavelength of the light 568. In this way, light from the light source 554 is prevented from undesirably being transmitted directly to the photodetector 558. In addition, the optical filter 564 can block light from passing through the optical filter 562 (and to the photodetector 558) that has a wavelength that is different than the light 568. In this way, the light emitted from the substate in the detection chamber 556 is the only light sensed by the photodetector 558.

In some non-limiting examples, including when each particle is genetic material, such as RNA, the detection chamber 556 (or a reaction chamber different than the detection chamber 556) can be used to multiply the particles, via an amplification process (e.g., a genetic amplification process). In some cases, the one or more reagents needed for the amplification process can be added to the detection chamber 556 (or reaction chamber), such as, by a pump (e.g., a pump in fluid communication with the one or more reagents). In other cases, the one or more reagents can be lyophilized, and can be positioned within the detection chamber 556. In this way, when the particle solution (e.g., that is concentrated) enters the detection chamber, the one or more lyophilized reagents dissolve in the particle solution. This can be advantageous in that the particle solution does not need to be diluted by the addition of the one or more reagents, which can improve the limit of detection (“LoD”) of the particles. In some cases, the one or more reagents can include one or more primers specific to a corresponding region of the genetic material of the microorganism or virus to be detected, a reverse transcriptase, a DNA polymerase, one or more primers specific to the analyte (e.g., a region of the genetic material that is the analyte), etc. In some configurations, the detection chamber 556 is configured to facilitate a loop mediated isothermal amplification (“LAMP”) reaction, a reverse-transcription loop-mediated isothermal amplification (“RT-LAMP”) reaction, etc. In some cases, the heater 560 (e.g., controlled by a computing device 570) can heat the particle solution (and the one or more reagents dissolved therein) to a temperature (e.g., greater than or equal to 65° C.) that allows the reaction to take place. In some configurations, using a LAMP reaction scheme, rather than a polymerase chain reaction (“PCR”) can be advantageous in that the particle solution can be maintained at a substantially constant temperature throughout the amplification process. In this way, the detection system 550, or the collecting system more broadly, does not need a thermal cycler to amplify the particles (e.g., genetic material), which can be bulky, require extensive thermal components, electronic circuits, etc.

In some non-limiting examples, the computing device 570, which can be in communication with the light source 554, and the photodetector 558, and can receive optical data from the photodetector 558 indicative of the light 568 interacting with the photodetector 558, during, for example, the amplification process. In some cases, the computing device 570 can utilize the optical data to generate an amplification curve (e.g., the measured fluorescence over time), which can be used by the computing device 570 to determine an initial amount of particles within the particle solution (or the concentrated particle solution). For example, the computing device 570 can determine a time (e.g., a time value) at which the amplification curve exceeds a threshold (e.g., a fluorescence threshold), which can correspond to an exponential (or linear) region of the amplification curve. Then, the computing device 570 can determine an initial amount of the particles within the particle solution (or the concentrated particle solution), based on the time (e.g., lower amounts of time correspond to larger initial amounts of the particles, because the particles begin exponentially multiplying sooner, and vice versa). In some configurations, the initial amount of particles can be used to inform (or otherwise) alert a user (e.g., by transmitting a warning to a user), or perform one or more remedial actions on the fluid surrounding the probe that collected the particles (e.g., the enclosed volume). For example, the computing device 570 can determine a multiplier that the particles were concentrated by from the particle solution to the concentrated particles solution (e.g., which can be retrieved from memory), an efficiency value of the ratio between the number of particles collected on the probe relative to a total number of particles within the fluid surrounding the probe (e.g., by retrieving the efficiency valve from memory), and a volume of an enclosed volume that the probe is positioned in (e.g., by retrieving the volume from memory). Then, the computing device 570 can determine an initial amount of particles within the particle solution by dividing the determined amount of particles by the multiplier, and can determine an initial amount of particles within the enclosed volume by applying the efficiency value (e.g., dividing, multiplying, as appropriate) to the initial amount of particles within the particle solution. Then, the concentration of the particles within the enclosed volume can be determined by dividing the initial amount of particles within the enclosed volume by the volume of the enclosed volume. In some cases, if the concertation of the particles exceeds a threshold value, then the computing device 570 can alert a user, implement one or more remedial actions, etc.

In some cases, the computing device 570 cannot determine a time, or in other words, an amplification curve does not form at least because the amount of particles are below the LoD of the detection system—even with the particles having been concentrated. In this case, the computing device 570 does not determine a presence of the particles within the surrounding fluid (e.g., with the particles being a pathogen), and the computing device 570 can alert, notify, etc., a user accordingly. In other cases, including when the computing device 570 does determine a time, the computing device 570 can determine a presence of the particles (e.g., a specific pathogen) within the surrounding fluid, and thus the computing device 570 can alert, notify, etc., a user, and can implement one or more remedial actions accordingly. In some configurations, while the heater 560 is illustrated as being outside of the chamber 552, in other configurations, the heater 560 can be positioned within the chamber 552.

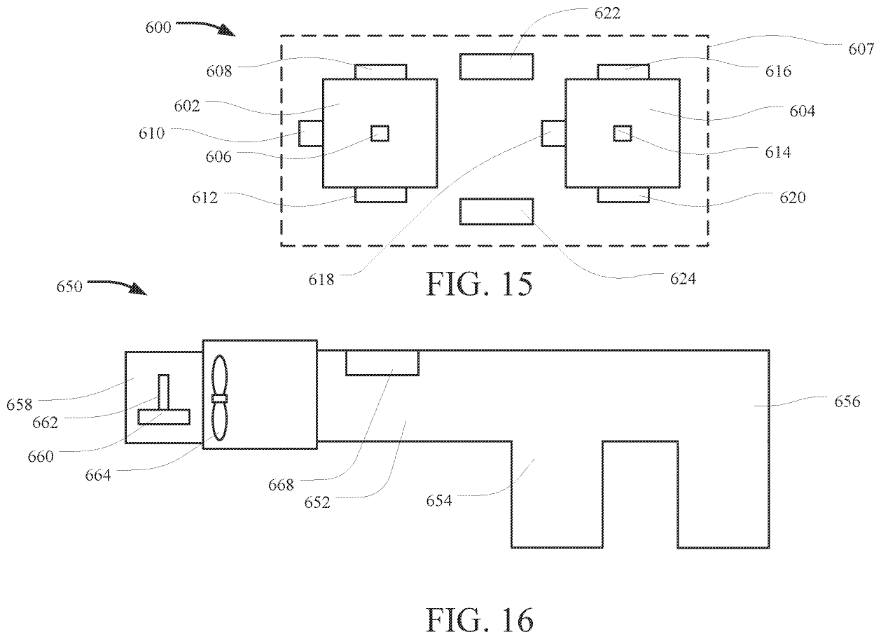

FIG. 15 shows a schematic illustration of an analysis system 600 that can include collecting systems 602, 604, each of which can be specific implementations of the other collecting systems described herein. Thus, the previously described collecting systems pertain to the collecting system 600 (and vice versa). Each collecting system 602, 604 can include one or more respective probes and one or more tractive devices to propel the respective collecting system 602, 604 around the enclosed volume 607. For example, the collecting system 602 can include a probe 606, and traction devices 608, 610, 612, each of which can be a wheel. As another example, the collecting system 604 can include a probe 614, and traction devices 616, 618, 620, each of which can be a wheel. In some cases, one or more of the traction devices can be powered by a motor (not shown) of the respective collecting system. In this way, each collecting system 602, 604 can be moved within the enclosed volume 607, which can advantageously prevent the a probe from being positioned in a dead spot of the enclosed volume 607, in which fluid does not flow (or flows at a much lower rate) thereby preventing adequate collecting of particles on the probe. In some cases, each collecting system 602, 604 can be configured to move within the enclosed volume 607 (e.g., in a random movement pattern). In some cases, having multiple collecting systems or a collecting system with multiple probes configured to be placed at different locations throughout the enclosed volume 607 can be advantageous in that a final particle concentration within the enclosed volume can be more accurate at least because additional locations within the enclosed volume 607 can be sampled. In addition, if one collecting system 602 does not detect the presence of the particles within the enclosed volume 607, but the collecting system 604 does detect the presence of the particles within the enclosed volume 607, the result from the collecting system 604 can be used to alert, notify, etc., a user, or implement one or more remedial actions. In this way, multiple collecting systems (or multiple probes) can more accurately detect the particles within the enclosed volume 607.

In some configurations, the analysis system 600 can include a computing device 622, and a disinfection system 624. The computing device 622 can be in communication with some or all of the components of the analysis system 600, as appropriate. For example, the computing device 622 can be in communications with the collecting systems 602, 604, and the disinfection system 624. In some cases, the computing device 622 can utilize data from both collecting systems 602, 604 to determine a concentration of particles within the enclosed volume 607. For example, the computing device 622 can receive a first concentration from the collecting system 602, receive a second concentration from the collecting system 604, and can utilize (e.g., combine) the first concentration with the second concertation to determine a resulting concentration. In some cases, the first concentration and the second concentration can be averaged to determine a resulting concentration, which can be used to evaluate whether or not the resulting concentration exceeds a threshold value.

In some non-limiting examples, the disinfection system 624 can be implemented in different ways. For example, the disinfection system 624 can include a pump, a fan, etc., that can drive fluid (e.g., air) into (or out of) the enclosed volume 607, include one or more disinfecting lights (e.g., an ultraviolet light (“UV” light) that are optically coupled to fluid within the enclosed volume 607 (e.g., or fluid that enters (or exits) the enclosed volume), one or more pumps that are in fluid communication with a disinfecting chemical (e.g., ethanol, such as 70 percent ethanol, etc.). Regardless of the configuration, the disinfection system 624 is configured to disinfect fluid within the enclosed volume 607, fluid that exits the enclosed volume 607 (e.g., that is then recirculated), fluid that enters the enclosed volume 607, etc. In some non-limiting examples, the enclosed volume 607 can be implemented in different ways. For example, the enclosed volume 607 can part of a building (e.g., a room of a building), a public building (e.g., a university, a school, a hospital, etc.), a vehicle (e.g., a bus, a car, etc.), an aircraft (e.g., a passenger airplane), a ship (e.g., a cruise ship), etc. Thus, the analysis system 600 (and each collecting system 602, 604) as broad applicability. In some cases, the computing device 622 can, based on the computing device 622 determining a presence or a concentration of the particles exceeding a threshold value, can activate the disinfection system 624, which can include, driving fluid into or out of the enclosed volume 607 (e.g., by activating a fan of the disinfection system 624), increasing the flow rate of fluid into (or out of) the enclosed volume 607 (e.g., by increasing the speed of the fan), activating a UV light that emits UV light at fluid within the enclosed volume 607 (or fluid that exits or enters the enclosed volume 607), increasing the amount of UV light directed at fluid, applying a disinfecting chemical to fluid within the enclosed volume 607 (or fluid that exits or enters the enclosed volume 607).

FIG. 16 shows a schematic illustration of an analysis system 650, which can be specific implementations of the analysis system 600. Thus, the analysis system 600 pertains to the analysis system 650 (and vice versa). The analysis system 650 can be a heating ventilation, and air conditioning (“HVAC”) system. Thus, the analysis 650 can include ducts 652, 654, 656, each of which can be in fluid communication with each other. As shown in FIG. 16, the ducts 654, 656 branch away from the duct 652, and each duct 654, 656 can provide fluid flow (e.g., air flow) to the same enclosed volume, or different enclosed volumes. The analysis system 650 can include a fluid intake 658 (e.g., an air intake) that can be in fluid communication with the one or more enclosed volumes that the analysis system 600 provides fluid to. For example, in the case of one or more enclosed volumes, each enclosed volume can be in fluid communication with the fluid intake 658, which can be advantageous in that fluid containing particulates is ensured to pass through the fluid intake 658. Thus, a probe of a collecting system is ensured to contact all the fluid (e.g., so that probe does not have to interact with a dead spot).

The analysis system 650 can include a collecting system 660 that is positioned within the fluid intake 658, or positioned downstream of the fluid intake 658. In addition, the analysis system 650 including the probe 662 can be fixed in place (e.g., stationary) during the collecting process, the analyzing process, etc. In other words, the position of the analysis system 650 and a reference portion of an enclosed volume can be the same. The collecting system 660 can include one or more probes 662. In some cases, including when the collecting system 660 includes multiple probes (or there one or more additional collecting systems each with one or more probe), each probe can be separated along the flow path of fluid that flows into the fluid intake 658 and through the duct 652. In this way, the usage of multiple probes can increase the accuracy of the concentration measurement—especially when sampling at different locations along the flow path. In some configurations, the analysis system 650 can include a fan 664, and a disinfection system 668. The fan 664 can be positioned within the duct 652 (or otherwise in fluid communication with the duct 652) and can drive fluid flow through the duct 652 and into the one or more enclosed volumes (e.g., via the ducts 654, 656). In addition, the disinfection system 668 can be positioned within the duct 652 (or otherwise in fluid communication with the duct 652) and can disinfect the fluid as the fluid flows pass the duct 652. For example, the disinfection system 668 can be a UV light source that can be activated to kill or otherwise denature particles (e.g., pathogens) that pass by through the UV light. In some non-limiting examples, the analysis system 650 can include other components of the HVAC system, including, for example, a heater, an air conditioning system, etc. In some cases, the disinfection system 668 can be a heater that heats the fluid to kill or otherwise denature the particles (e.g., pathogens) that pass by the heater (or component heated by the heater).

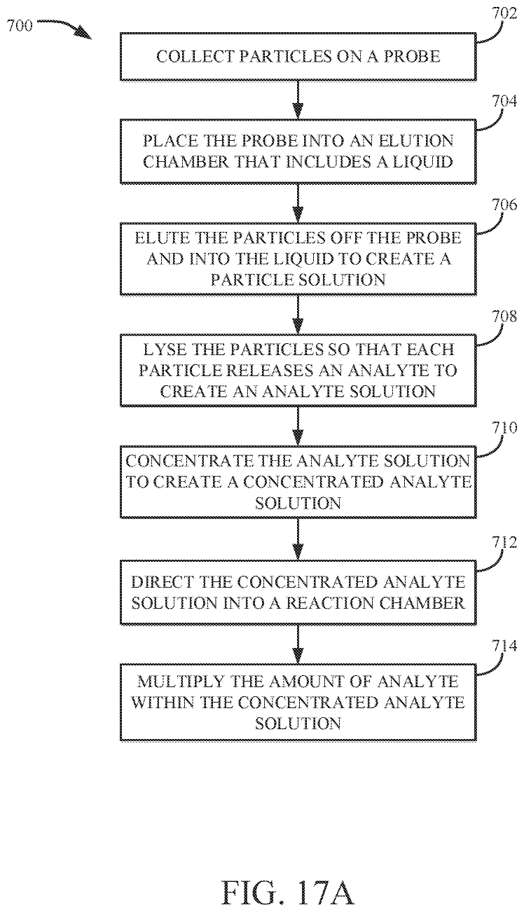

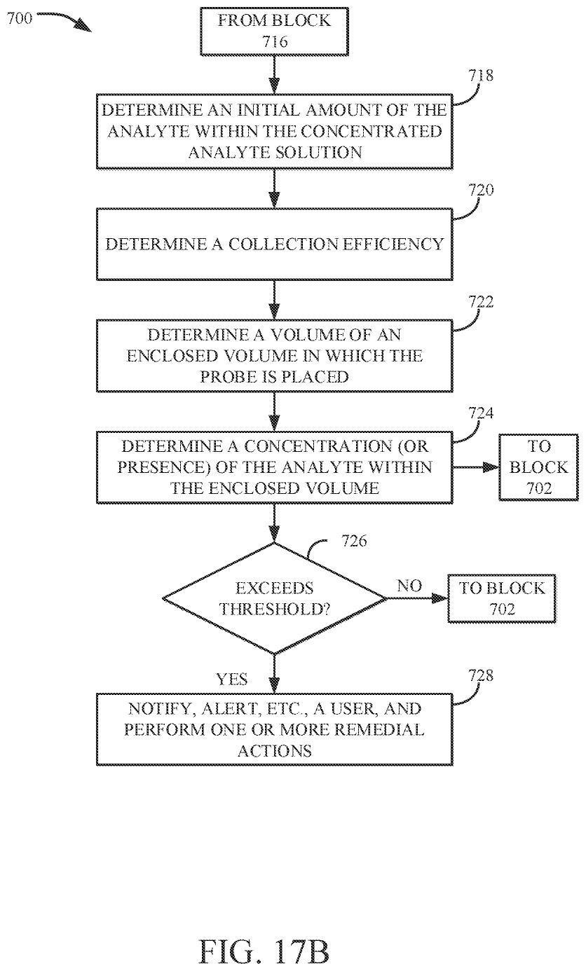

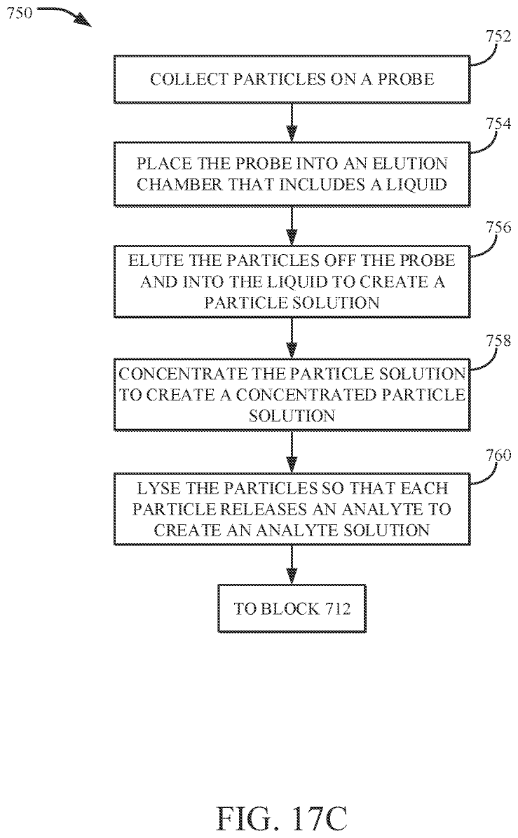

FIG. 17 shows a flowchart of a process 700 for collecting and analyzing particles. The process 700 can be implemented using any of the collecting systems, any of the analysis systems, etc., as appropriate. In addition, some or all of the blocks of the process 700 can be implemented using one or more computing devices, as appropriate.

At 702, the process 700 can include a computing device collecting particles on a probe. For example, this can include a computing device causing the probe to be electrically grounded (e.g., by activating a switch), and causing one or more ionizers to ionize the surrounding fluid that includes particles positioned therein (e.g., aerosolized pathogens).

At 704, the process 700 can include a computing device placing the probe into an elution chamber that includes a liquid (e.g., deionized water). In some cases, this can include a computing device causing an actuator (or a robotic arm) to move the probe into the elution chamber. In some configurations, this can include a computing device placing the probe into contact with the liquid.

At 706, the process 700 can include a computing device eluting the particles off the probe and into the liquid to create a particle solution, using an elution device. In some cases, the block 706 can include mixing the liquid so that the liquid contacts the surface of the probe to elute the particles off the probe.

At 708, the process 700 can include a computing device lysing the particles so that each particle releases an analyte to create an analyte solution. In some cases, this can include passing the particle solution through a component that is heated by a heater. In some cases, the analyte can be a genetic material. For example, the particles can be each be a specific pathogen (e.g., SARS-Cov-2) and the particles can be lysed to release the genetic material of the pathogen into the liquid (e.g., the genetic material being the analyte).

In some configurations, the block 708 can be implemented before the block 706. For example, while the probe is positioned within the elution chamber, the liquid can be heated to a temperature (e.g., 90 degrees Celsius) to lyse the particles so that the particles release respective analytes. In some cases, these analytes can be charged, and thus a computing device can cause the probe to be charged, or can cause an electrode to be charged. In this way, the charged analyte can be electrically forced away from the probe. In some cases, while the block 708 has described lysing the particles using heat, which can be advantageous (e.g., can be quick), in other cases, the particles can be lysed by using a chemical. For example, including when the particles are spores (e.g., a bacterial spore, a mold spore, etc.), the chemical can be added to the particle solution to cause the particles to lyse their genetic contents. In some cases, the chemical can be one of the chemicals of the ZymoBIOMICS DNA Miniprep, available at Zymo Research, DNeasy® Power Soil®, available at Qiagen

At 710, the process 700 can include a computing device concentrating the analyte solution to create a concentrated analyte solution. For example, a computing device can cause one or more electrodes to be electrically charged, and can cause a pump to direct fluid at the analyte solution. The one or more electrodes can electrically force the analytes closer together to create a concentrated analyte solution. In addition, the pump can force the liquid through a barrier, but the barrier can block the analyte from passing through the barrier thereby creating a concentrated analyte solution. In some cases, the concentrated analyte solution can be at least 1.5 times, at least 2 times, at least 3 times, at least 4 times, at least 5 times, at least 5.4 times, etc., the concertation of the analyte of the analyte solution.

At 712, the process 700 can include a computing device directing the concentrated analyte solution (or the analyte solution) to a reaction chamber (e.g., that can be a detection chamber) by using, for example, a pump.

At 714, the process 700 can include a computing device multiplying the amount of analyte within the (concentrated) analyte solution. In some cases, this can include a computing device adding one or more reagents to the reaction chamber. In other cases, the reaction chamber can be preloaded with the one or more reagents. For example, the one or more reagents can be lyophilized and can be positioned within the reaction chamber. Thus, when the (concentrated) analyte solution is directed into the reaction chamber, the process can include dissolving the one or more reagents that are lyophilized into the (concentrated) analyte solution. In some cases, this can include a computing device causing a heater to heat the (concentrated) analyte solution to a temperature that facilitates multiplying of the analyte. In some cases, during the multiplying process, the analytes can multiple without lowering the temperature substantially below the temperature, or raising the temperature substantially above the temperature (e.g., to a temperature in which the analyte that is DNA denatures).

At 718, the process 700 can include a computing device determining an initial amount of the analyte within the (concentrated) analyte solution. For example, this can include a computing device determining an amplification curve, and using the amplification curve to determine the amount of the analyte within the (concentrated) analyte solution. In some cases, if the analyte solution is concentrated, then a computing device can determine the amount of analyte within the concentrated analyte solution, and can determine the initial amount of the analyte within the analyte solution (e.g., prior to being concentrated) by dividing the amount of analyte by a concentration multiplier (e.g., the percentage a given analyte solution is concentrated by, which can be specific to the concentrator used).

At 720, the process 700 can include a computing device determining a collection efficiency (e.g., an efficiency value of the ratio between the number of particles collected on the probe relative to the total number of particles within the fluid surrounding the probe). In some cases, the collection efficiency can factor in the efficiency of the amount of particles collected in a liquid verses the total amount collected on the probe (e.g., there are some losses because in some cases not all particles can be removed from the probe).

At 722, the process 700 can include a computing device determining a volume of an enclosed volume in which the probe is located. In some cases, this can include a computing device retrieving the value from memory (e.g., the value having been previously received by the computing device). In some cases, this can include a computing device receiving the volume from a user input (e.g., a user manually entering in the volume).

At 724, the process 700 can include a computing device determining a concentration (or presence) of the analyte within the enclosed volume. In some cases, this can include determining a presence of the analyte (e.g., an analyte to be detected), which can be based on the presence of a concentration determined at the block 720, the presence of an amplification curve, etc. In some cases, a computing device can determine that the analyte is not present within the surrounding fluid of the enclosed volume, based on the amplification curve being present, or a point of on the amplification curve (e.g., a fluoresce value) being below a threshold value. In some cases, if at the block 724, a computing device determines that an analyte is not present within the analyte solution, the process 700 can proceed to the block 702 to collect additional particles on the probe. If, however, at the block 724, a computing device determines that an analyte is present within the analyte solution, the process 700 can proceed to the block 724 (or the process 700 can proceed to the block 726). In some non-limiting examples, a computing device can combine (e.g., average) multiple concentrations of an analyte within an enclosed volume to yield a determined concentration.

At 726, the process 700 can include a computing device determining whether or not the determined concentration of the analyte (e.g., within the enclosed volume) exceeds a threshold value. If at the block 726, a computing device determines that the determined concentration of the analyte exceeds the threshold value (e.g., is greater than), the process 700 can proceed to the block 728. If, however, at the block 726, the process 700 determines that the determined concentration of the analyte does not exceed the threshold value (e.g., is less than the threshold value), then the process 700 can proceed back to the block 702.

At the block 728, the process 700 can include a computing device notifying, alerting, etc., a user (e.g., based on the presence of the analyte or the concentration of the analyte exceeding a threshold value). In some cases, this can include a computing device presenting on a display an alert indicating a warning to a user. In other cases, this can include a computing device alerting a user to block off access to the enclosed volume (e.g., a room). In some configurations, the block 728 can include a computing device performing one or more remedial actions (e.g., based on the presence of the analyte or the concentration of the analyte exceeding a threshold value). In some cases, the one or more remedial actions can include activating a heater to heat of a first fluid (e.g., that is entering, exiting, or positioned within the enclosed volume), increasing the thermal output of the heater to the first fluid, activating a fan to increase fluid flow into (or out of) the enclosed volume, increasing the flow rate of fluid flow into (or out of) the enclosed volume, activating a disinfection system (e.g., a UV light) to disinfect the first fluid, increasing the power output to a disinfection system (e.g., increasing the power provided to the UV light thereby increasing the amount of UV light) that disinfects the first fluid, etc.

In some non-limiting examples, the process 700 can include a computing device determining that a pathogen is capable of being aerosolized that has previously been unknown to be capable of being aerosolized, based on the computing device determining a presence of the pathogen (e.g., within the solution). In some cases, a computing device can proceed to the block 728 to notify, alert, etc., a user, based on the computing device determining that the pathogen is capable of being aerosolized. In this way, the computing device can readily disseminate critical information of a potential pathogen, which can inform the general public of the properties of the pathogen to better allow the general public to better evaluate their risks regarding the pathogen in public settings. Thus, the process 700 and the collecting systems herein, can help with the discovery and rapid dissemination of crucial information regarding the ability of certain pathogens, biological threats, etc., to be transmitted via aerosols.

FIG. 17C shows a flowchart of a process 750 for collecting and analyzing particles, which can be implemented using any of the collecting systems, analyzing systems, etc., described herein. In addition, some or all of the blocks of the process 700 can be implemented using one or more computing devices.

At 752, the process 752 can include a computing device collecting particles on a probe, which can be similar to the block 702 of the process 700. At 754, the process 700 can include a computing device placing the probe into an elution chamber that includes a liquid, which can be similar to the block 704 of the process 700.

At 754, the process 700 can include a computing device eluting the particles off the probe and into the liquid to create a particle solution, which can be similar to the block 706 of the process 700. In some cases, the liquid can be mixed to elute the particles off the probe and into the liquid. In some configurations, when the particles are charged (e.g., the particles having a zeta potential that is negative or positive, such as, for example, when the particles are viruses and have a capsid that has a zeta potential that is positive), the probe (or an electrode) can be charged to repel (or attract) the particles off the probe into the liquid and away from the surface of the probe.

At 756, the process 750 can include a computing device concentrating the particle solution to create a concentrated particle solution, which can be similar to the block 710 of the process 700. In some cases, this can include a computing device causing one or more electrodes to be electrically charged to force the particles closer together, thereby concentrating the particle solution. In some cases, this can include a pump (e.g., a piston pump) driving the particle solution against a semi-permeable barrier that is permeable to the liquid, but is impermeable to the particles. In this way, the particle solution can be concentrated into a concentrated particle solution.

At 760, the process 750 can include a computing device lysing the particles so that each particle releases an analyte (e.g., genetic material) to create an analyte solution, which can be similar to the block 708. For example, when the particles are spores, a chemical can be added to the concentrated particle solution (or a particle solution) to lyse the spores so they release their genetic contents (e.g., the analyte). In some configurations, concentrating the particles before lysing can be advantageous in that the genetic material (e.g., the RNA), may be less likely to be damaged during a concentrating process (e.g., because the capsid, or the spore protects the genetic material from damage during an elution process).

In some embodiments, once the analyte solution has been created (e.g., at the block 760), the process 750 can proceed to the block 712 of the process 700, to, for example, multiply the amount of analyte, analyze the analyte (e.g., determine a concentration of the analyte), determine whether or not a threshold is exceeded, etc. In some configurations, if a computing device determines that the analyte concentration does not exceed a threshold, then the process 750 can proceed back to the block 752 (e.g., to collect additional particles on the probe).

### EXAMPLES

The following examples have been presented in order to further illustrate aspects of the disclosure, and are not meant to limit the scope of the disclosure in any way. The examples below are intended to be examples of the present disclosure and these (and other aspects of the disclosure) are not to be bounded by theory.

There has been an increased interest in ways to monitor indoor environments for on-site detection of viruses, especially viruses found in aerosols in the air of indoor environments.

The Coronavirus disease 2019 (“COVID-19”) pandemic currently has caused more than 406 million infections and more than 5.7 million deaths worldwide. Severe acute respiratory syndrome coronavirus 2 (“SARS-CoV-2”) is the causative agent of this disease. Airborne transmission has been identified as the dominant route for the spread of COVID-19. In particular, viable SARS-CoV-2 has been recovered from aerosols in hospital rooms of COVID-19 patients, indicating potential aerosol-induced infection. As a consequence of the risk of infection through virus-loaded aerosol, the World Health Organization (“WHO”) updated the guidelines for infection prevention. Also, the Center for Disease Control and Prevention (“CDC”) has officially recognized aerosols as a potential transmission mechanism. A commentary signed by 239 researchers from the scientific and medical community around the globe are advocating preventive measures to mitigate this route of airborne transmission. Thus, the ability to perform on-site detection of aerosolized SARS-CoV-2 in indoor environments could be another way to prevent the spread of this disease (or other diseases that are transmitted through aerosolized pathogens).

Currently, no system exists for on-site monitoring and detection of SARS-CoV-2 present in the air of indoor environments. The system described herein integrates an air sampler that uses electrostatic precipitation with a highly sensitive RNA detection method (e.g., RT-LAMP) to enable detection of SARS-CoV-2 in the aerosol in quantities comparable to those found for influenza virus during flu season in closed spaces.

Currently existing high-accuracy methods to detect SARS-CoV-2 require a sample of physiological material (from nose or throat), and hence cannot be used for aerosol virus detection. By contrast, the method described herein allows sampling of virus from the air in closed environments by using an electrostatic precipitation-based air sampler. Also, the method uses RT-LAMP, which retains the high sensitivity of RT-qPCR, but does not require a thermal cycler (e.g., the thermal cycler can prevent on-site detection because of the bulkiness of the thermal cycler). In addition, RT-LAMP is a one-step reaction that can be completed by heating a sample to 65° C.

The system for monitoring and detecting virus present in the air of closed environments can be used broadly in both public spaces and private homes or businesses. The system acts as an early warning of the presence of potentially harmful concentrations of SARS-CoV-2 (or other viruses) in the air. As such, it can trigger an alarm to alert surrounding people that an evacuation or an air-exchange may be required for safety. Thus, this system is applicable to a wide range of application uses including use in hospitals, clinics, care-homes, schools, colleges, airports, cruise ships, other public places, other places where an aerosolized virus can spread, etc.

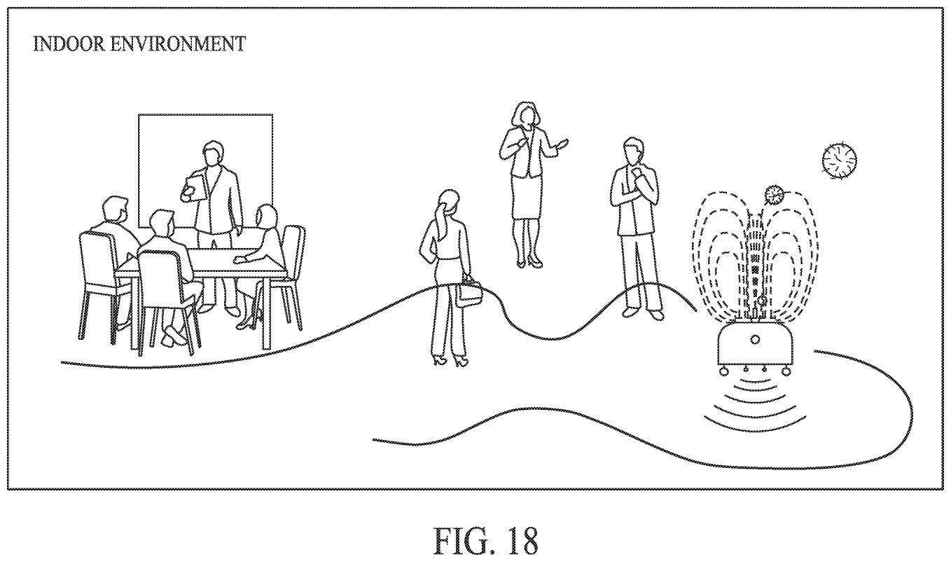

FIG. 18 shows a system that can perform continuous monitoring of an indoor environment of interest for on-site detection of virus in aerosol. For example, this system can be for on-site detection of virus-loaded aerosol, focusing specifically on detection of SARS-CoV-2, but the systems and methods can be applicable to on-site detection of any airborne biological particles (e.g., microorganisms, viruses, etc.). Specifically, the air sampler of the system uses electrostatic precipitation (“ESP”) for sampling (virus-loaded) aerosols. This air sampler is coupled with a highly sensitive chemical nucleic acid detection method called reverse-transcription loop-mediated isothermal amplification (“RT-LAMP”). This method, different from the RT-PCR method used in current laboratory COVID tests, can be performed on-site since an isothermal reaction for RT-LAMP exempts from the time lost in thermal cycles in which heating and cooling are required for conducting a RT-PCR reaction and no complicated and bulky thermal cycler is required. In other words, using RT-LAMP (rather than RT-PCR) can be faster as unlike RT-PCR, RT-LAMP does not require the solution to be heated and cooled to different temperatures, which requires more time.

Briefly, all virus particles collected on the probe of the ESP sampler are washed off in a small volume (50 μL) of de-ionized water. A 5 minute pre-heating step at 90° C. extracts, for example, the RNA genome from the RNA virus. RT-LAMP is then performed in a one-step reaction using primers specific to the virus to be detected. RT-LAMP converts RNA to cDNA and amplifies DNA with an isothermal reaction at 65° C. for about 25 minutes. A portable fluorescent signal-readout module continuously monitors the DNA amplification to determine the presence of viral genome in real-time.

The system can continuously sample the environment and can interrogate it periodically to determine the presence of the virus of interest. The system can eventually send out an early warning when airborne virus concentration exceeds a defined threshold. In some cases, the interrogation can take 30-45 minutes.

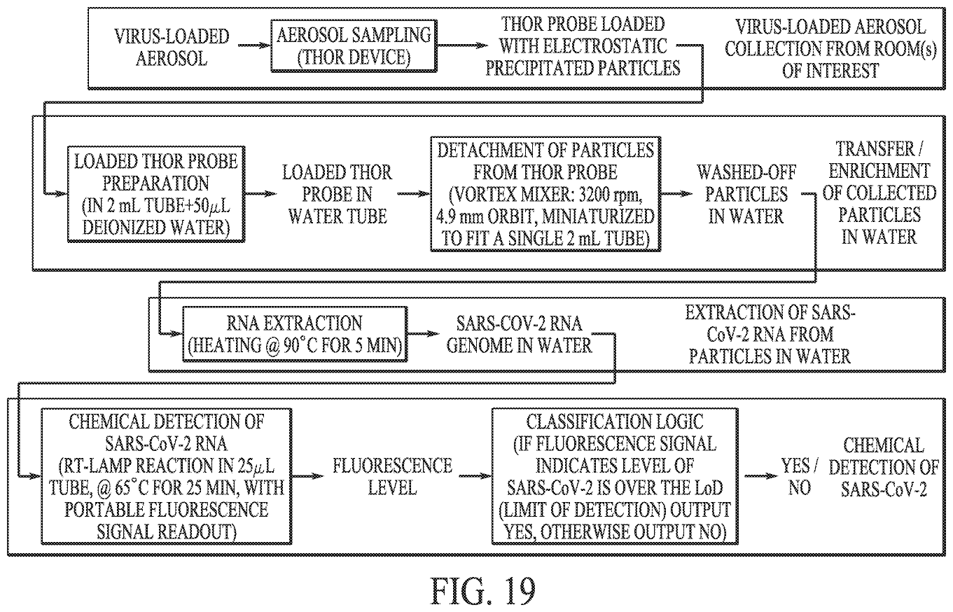

FIG. 19 details the process for on-site detection of virus in aerosol. At a high-level, the system first collects the aerosol from room(s) of interest on the metal probe of the sampling device. Then, the collected particles are detached from the probe and concentrated in water using a vortex mixer. Once in water, viral RNA is extracted by a 5-minute heating step at 90° C. After RNA extraction, the system performs chemical detection by a one-step RT-LAMP reaction at 65° C. A portable fluorescence reader monitors fluorescence level produced by RT-LAMP reaction for 25 minutes. If within the 25 minutes the fluorescence level is above a pre-set threshold, then the system outputs “YES”, otherwise it outputs “NO”.

Virus-loaded aerosol collection and transfer/enrichment of collected particles in water is described in Section 2.1 along with experimental quantification of overall collection efficiency from air to water. RNA extraction is described in Section 2.2 along with its efficiency. Chemical detection of SARS-CoV-2 via RT-LAMP is described in the Section 2.3, along with experimental quantification of the limit of detection (LoD) and statistics of false positives/negatives. The portable fluorescence reader is briefly described in Section 2.4.

### Section 2.1. Aerosol Sampling and Transfer/Enrichment of Collected Particles in Water

A process allows collection, from the air into water, of a concentration of particles of interest that is above the LoD of the RT-LAMP chemical detector, when the concentration of such particles in the air is within the typical range of influenza virus found airplanes/day care centers during flu season. Assuming that concentrations in air of SARS-CoV-2 are similar to those of influenza virus during flu season, the process can detect practically relevant amounts of SARS-CoV-2 in the air.

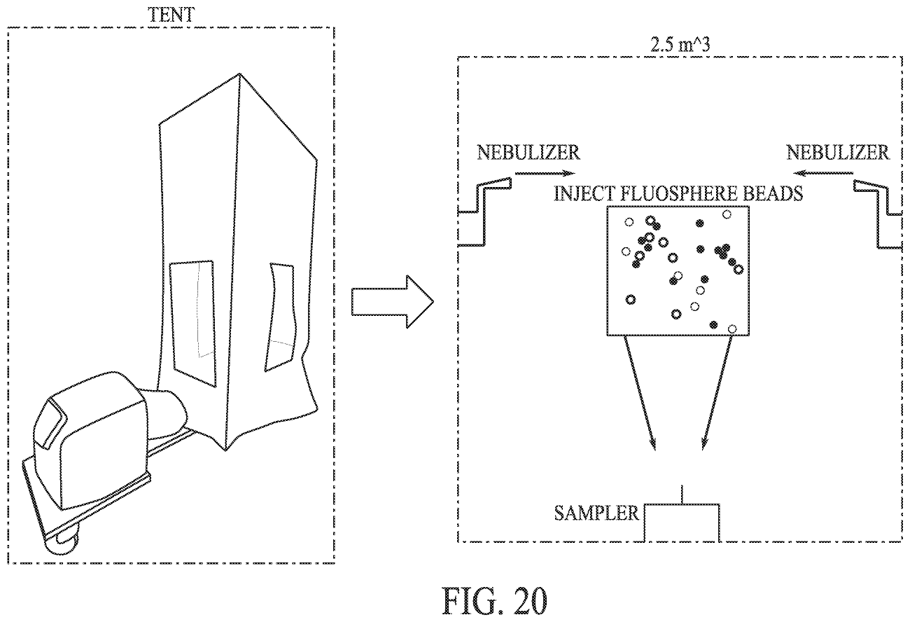

Referring to FIG. 20, a probe device was created for aerosol sampling. The probe device is an aerosol sampler based on corona discharge ionization and electrostatic precipitation. After the probe device has been sampling the air in desired room(s) by walking around the space, the probe device becomes loaded with particles of interest, such as potential viral particles. The probe device is then detached and placed into a 2 mL tube along with, for example, 50 μL of de-ionized water. Particles are detached from the—probe by using a vortex mixer for a few seconds. The smaller volume of water chosen allows for a higher concentration of the particles in water. The quantitative characterization of collection efficiency of this process is detailed next.

Probe device description: The probe device is an aerosol sampler based on corona discharge ionization and electrostatic precipitation. When the probe device is on (e.g., receiving electrical power), a corona discharge occurs in correspondence to the carbon brushes tips, ionizing the air molecules around them. The aerosol particles are, in turn, ionized and then, due to the electric field between the ionizers (−20 kV) and the collector (grounded), they move towards the collector and, when they reach it, they lose their charge.

Experimental setup to emulate on-site collection: The experiments to characterize the overall collection efficiency were conducted inside a containment cube (Abetement Technologies, AG3000MCCK) as shown in FIG. 20. Solutions of 1 μm diameter FluoSphere (Thermo Fisher, F13081), emulating viral particles, were properly diluted in filtered water to the desired concentrations and they were introduced in the tent through four nebulizers (Omron, NEC801) located at the four top corners of the tent. The ESP sampler was located in the tent in a central position, 20 cm from the ground and 1.5 meters from the nebulizers. At the end of the sampling phase, the collector was transferred in a 2 mL microcentrifuge tube and filtered water was added to the tube with the collector inside. Then, after vortexing the tube, the collector was removed thorough a magnet and a defined amount of CountBright Absolute Counting Beads (Invitrogen, C36950) was added to the sample left in the tube and analyzed using a BD Accuri C6 flow cytometer (BD Biosciences, San Jose, CA).

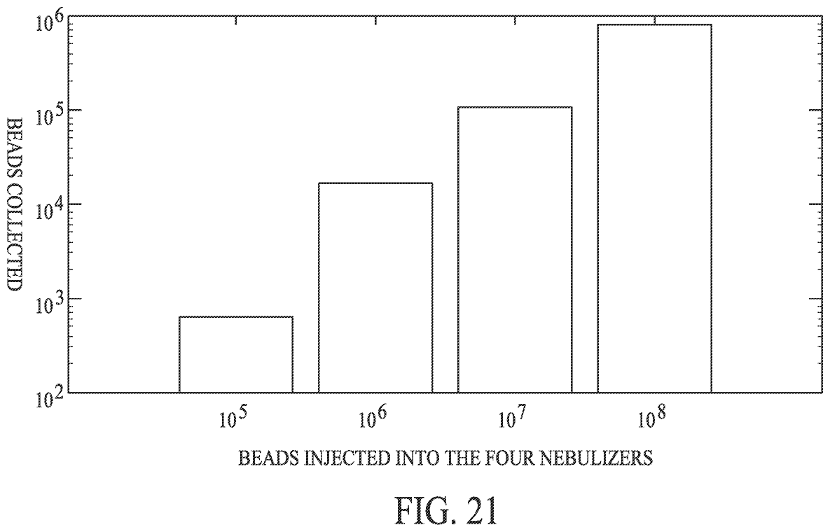

Evaluation and optimization of collection efficiency: To gauge the efficiency of the collection process from air to water, a titration experiment was performed to determine the ratio between the total number of particles placed in the nebulizers and the particles collected in water, for example, using a de-ionized water volume of 250 the amount of water that completely covers the probe in the 2 mL tube. The results are shown in FIG. 21, which indicate that the ratio of beads collected by the ESP sampler in water over beads introduced into the nebulizers is roughly equal to:

$$r = \frac{number of beads collected in water}{number of beads introduced in nebulizer} \approx 0.01 .$$

It is noted that the number of beads introduced in the nebulizer is larger than the number of beads injected in the tent. Thus, the reported value of r is an under-estimation of the ratio between the number of particles collected in water and those present in the air.

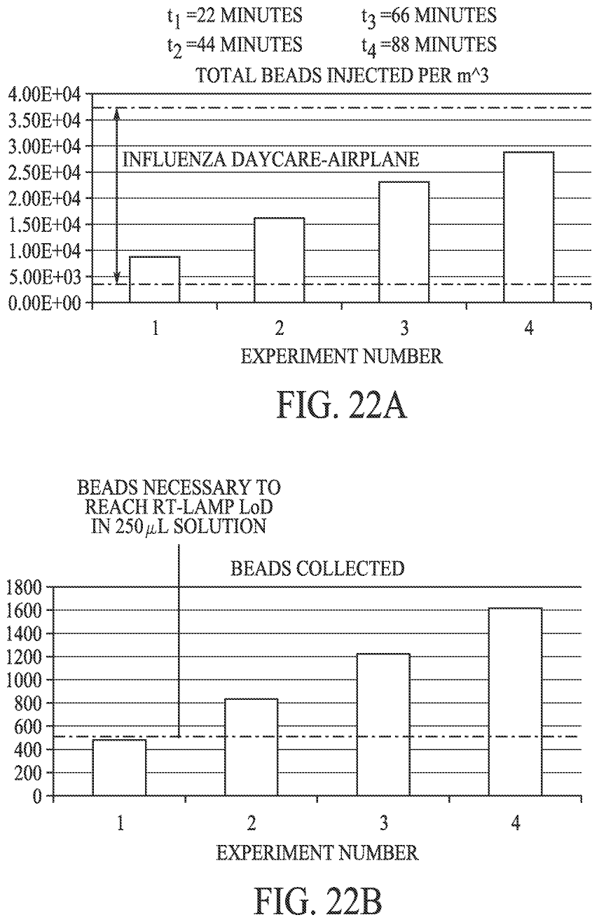

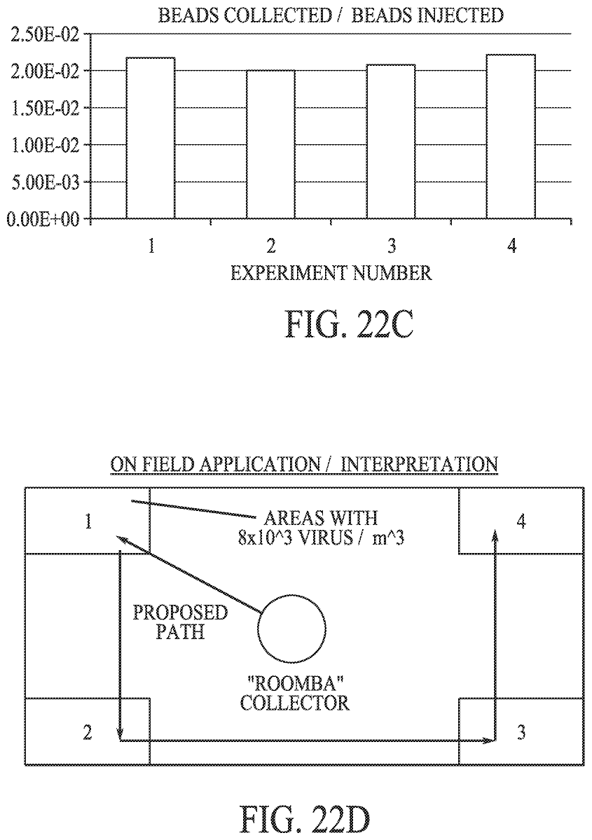

The second experiment that was performed had the objective of testing the sampling and collection efficiency in an environment that resembles on-field conditions, such as having the device roaming in a closed room with one or more sick people (see FIG. 22A-D). To this end, a continuous but small rate of nebulization was considered (simulating someone speaking or breathing) and a simultaneous collection (device roaming and collecting around the room). In particular, four experiments were conducted that can be described as follows:
Experiment 1: turn on the first nebulizer for 22 minutes.
Experiment 2: turn on the first nebulizer for 22 minutes, then turn off and turn on the second nebulizer for 22 minutes.
Experiment 3: turn on the first nebulizer for 22 minutes, then turn off and turn on the second nebulizer for 22 minutes, then turn off and turn on the third nebulizer for 22 minutes.
Experiment 4: turn on the first nebulizer for 22 minutes, then turn off and turn on the second nebulizer for 22 minutes, then turn off and turn on the third nebulizer for 22 minutes, then turn off and turn on the fourth nebulizer for 22 minutes.

The probe device was kept on for the entire experiment (i.e., t_{nebulization}=t_{sampling}). For experiments 1-4 the total number of FluoSpheres placed inside the nebulizers approximately corresponds to a concentration inside the containment cube as indicated by the values shown in FIG. 22A-D. These concentrations are within typical values found in airplanes/day care centers during influenza season, as indicated by the dashed lines.

The results of these experiments are shown in FIG. 22B and 22C. Based on these results it was concluded that for an expected on-field viral concentration, increasing the sampling time will increase the amount of particles collected. This implies that, if there is one or more sick people in a room, the probe device will be able to collect an amount of particles equal or higher than the RT LAMP LoD after a sufficiently long time tₘᵢₙ. Given that the RT-LAMP LoD with the primers is given by 50 copies per 25 μL reaction (i.e., 2 copies per μL), the minimum number of beads collected in 250 μL water that can be detected by the RT-LAMP is given by 500 copies. As a consequence, it is noted that the minimum concentration of particles in the air that can be detected is given by 16500 beads per cubicmeter, after collecting for tₘᵢₙ=44 minutes. The particles were injected constantly for the entire experimental time (44 minutes), meaning that the instantaneous concentration in the tent should be lower than 16,500 beads per cubicmeter.

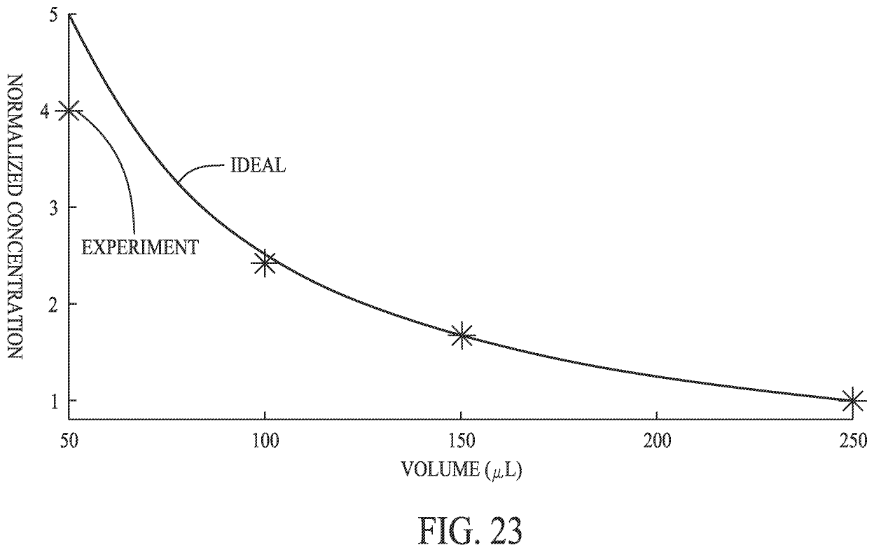

In order to optimize the process to enable detecting a lower concentration of particles in the air, the volume of de-ionized water added was decreased to the 2 mL tube. The motivation behind these experiments was that, in principle, by reducing the amount of water the concentration of particles collected could be increased, as long as this reduction would not adversely affect the mechanics of the removal process. In particular, one experiment was conducted (beads injected≈2×10⁵ per cubicmeter; simultaneous nebulization and collection with t_{nebulization}=t_{collection}=25 minutes) four times, reducing each time the volume of water added in the tube. The volumes of 50 μL, 100 μL, 150 μL and 250 μL were considered as the volumes of de-ionized water to add to the 2 mL tube. The results are shown in FIG. 23. Indeed, by reducing the volume we were able to increase the concentration of collected particles by up to four times.

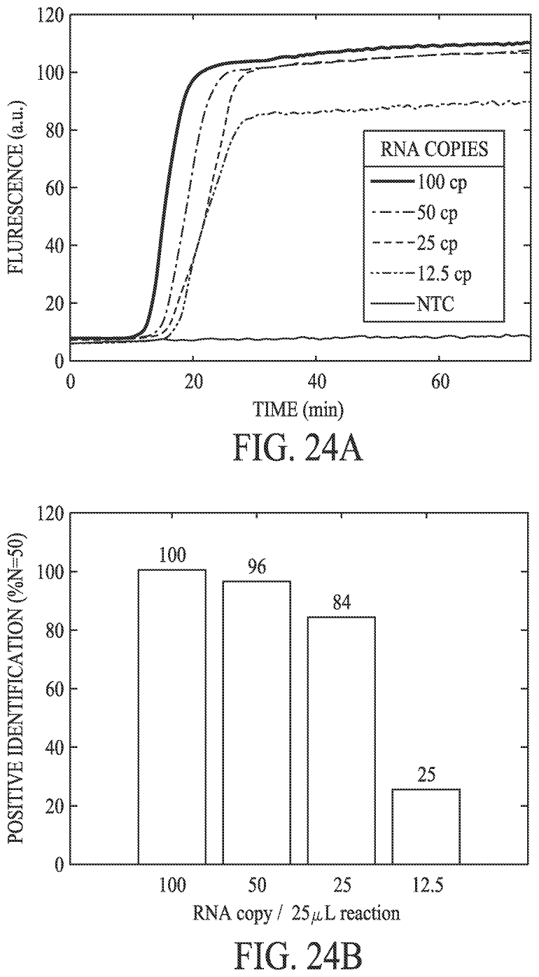

As a consequence, since the the RT-LAMP LoD is given by 500 copies per 250 μL (that is, 2 copies per μL), if the volume of water is reduced by 5 (that is, from 250 μL to 50 μL) in the experiment associated to FIG. 24B, the minimum number of beads collected that can be detected by the RT-LAMP is given by 2×250/4 =125. Thus, with 50 μL, of de-ionized water in the vortex mixer, the smallest concentration of particles in the air that can be detected by RT-LAMP is estimated to be about 8740 bead per cubicmeter, after collecting for tₘᵢₙ=22 minutes. This number is on the low end of the typical concentration of influenza virus in the air of closed spaces during flu season. Thus, sufficiently low concentration of SARS-CoV-2 should be detectable in the aerosol when sampling for 20-30 minutes.

### Section 2.2 RNA Extraction

To bypass a laborious RNA extraction step, a pre-heating process at 90.0 for 5 minutes was chosen as a proven high-efficiency method to release the RNA genome from the viral capsid.

### Section 2.3 Chemical Detection of Viral RNA

An RT-LAMP reaction was chosen as the chemical detection method of, for example, viral RNA, since it can be performed through a one-step reaction at 65° C., without the need of a thermal cycler. The RT-LAMP reaction uses the WarmStart LAMP 2× Master Mix kit (New England Biolabs, catalog E1700) that already includes a fluorescent dye (e.g., a nucleic acid stain), and a 10× stock of the combined primer sets to set up a 25 μL reaction with nuclease-free water (Zymo Research, catalog W1001) in a well of the 96-well plate (Axygen, catalog number PCR-96-LC480-W). The primer set N2 and the primer set Orf1a-HMSe target the N gene and a nonconserved region of the SARS-CoV-2 Orf1a gene, respectively. By using two primer sets, the limit of the detection (LoD) improved by 2-fold, which is consistent with other work. The RT-LAMP method can be tailored to any other virus of interest by simply changing the primer set. Table shows different primers that can be used to target the RNA of the SARS-CoV-2 virus.

TABLE 1
The primer sets of RT-LAMP.

| | |
|---|---|
| Primer | |
| Set | Sequence |

N2

| | |
|---|---|
| F3 | ACCAGGAACTAATCAGACAAG |
| B3 | GACTTGATCTTTGAAATTTGGATCT |
| FIP | TTCCGAAGAACGCTGAAGCGGAACTGATTACAAACATTGG |
| | CC |
| BIP | CGCATTGGCATGGAAGTCACAATTTGATGGCACCTGTGTA |
| LF | GGGGGCAAATTGTGCAATTTG |
| LB | CTTCGGGAACGTGGTTGACC |

Orf1a-HMSe

| | |
|---|---|
| F3 | CGGTGGACAAATTGTCAC |
| B3 | CTTCTCTGGATTTAACACACTT |
| FIP | TCAGCACACAAAGCCAAAAATTTATTTTTCTGTGCAAAGG |
| | AAATTAAGGAG |
| BIP | TATTGGTGGAGCTAAACTTAAAGCCTTTTCTGTACAATCC |
| | CTTTGAGTG |
| LF | TTACAAGCTTAAAGAATGTCTGAACACT |
| LB | TTGAATTTAGGTGAAACATTTGTCACG |

In order to determine the LoD and the rate of false positive and false negatives, the RT-LAMP reaction was ran in the lab using synthesized SARS-CoV-2 RNA target, incubated with a constant temperature at 65° C. In order to assess false positive rate, the reaction was ran for 1.5 hours. In order to monitor in real-time fluorescence of DNA dye, the one-step reaction was ran in the Roche LightCycler 480.

N=50 biologically independent experiments were conducted to determine the LoD of the optimized RT-LAMP condition described above. The LoD was defined as the number of copies per 25 μL of viral RNA that can be detected with at least 95% positive rate (see FIG. 24). The results are as follows:
limit of detection (LoD): 50 copies per 25 μL reaction (i.e., 2 copies per μL).
time to detect the LoD: 14.4±2.1 minutes. The 95% confidence interval is between 13.8 and 15 minutes.
positive percentage of identification of 50-copy RNA: 96%.

To setup a 25 μL reaction of RT-LAMP, it is possible to setup from two different formats. Starter format 1: liquid reagents: The volume of RT-LAMP reagents is 12.5 μL. The volume of RNA sample should be 12.5 μL. The combined 25 μL solution is ready to conduct RT-LAMP reaction in the signal read-out device. Starter format 2: lyophilized reagents: The benefit of this format is to maximize the RNA sample volume in a reaction. The RT-LAMP reagents are lyophilized and stored in the compartment to be conducted RT-LAMP reaction. The volume of RNA sample should be 25 μL to re-hydrate the RT-LAMP reagents and proceed the subsequent RT-LAMP reaction.

### Section 2.4 Fluorescent Signal Readout

Detection of successful amplification of the RT-LAMP target amplicon relies on semi-quantitative detection of DNA dye fluorescence. DNA dye is an intercalating dye that becomes fluorescently active upon interaction with double-stranded DNA (dsDNA). The excitation peak of, for example, SYTO9 is approximately 493 nm and the emission peak is approximately 503 nm. A system including an excitation source, optical chamber containing DNA dye and dsDNA-rich RT-LAMP product, photodetector, and optical filters is employed to detect fluorescent light and maximize the recovered signal.

The RT-LAMP reaction proceeds in a temperature-controlled optical chamber that is monitored in real-time. An LED is positioned immediately above the optical chamber to excite the sample through a clear aperture. The geometry of the optical chamber is to be optimized to recover the maximum fluorescent signal-to-noise ratio, while considering the impacts of geometry on non-template RT-LAMP amplification. The spectrum of the current-driven 5W LED is centered around, for example, 493 nm, and a lowpass filter is applied to the light path immediately after emission from the LED to eliminate wavelengths that interfere with detection of emission light. After exciting the sample, fluorescent emission light exits the optical chamber through the clear aperture. The emission light passes through a highpass filter excluding excitation light. Filtered emission light is received by a photodiode-based photodetector. The apparatus is sealed in a dark chamber.

Digital counts obtained from the photodetector are mapped to arbitrary units of fluorescence intensity by a microcontroller. Time-series data is used to construct real-time curves of fluroescence vs. time in the standard manner used in RT-qPCR (see FIG. 24). Amplification threshold times are defined using the second derivative method, a standard quantification analysis for RT-LAMP.

In summary, a system is proposed for on-site monitoring and detection of, for example, SARS-CoV-2, and of other viruses more generally, present in the air of closed environments. This system integrates an air-sampling device with a highly sensitive, for example, RNA, chemical detection method in order to enable detection of relevant concentrations of virus in the air.

### Section 3. Advantages, Improvements, and Applications

Currently, no system exists for on-site monitoring and detection of SARS-CoV-2 present in the air of indoor environments. This system integrates an air sampler that uses electrostatic precipitation with a highly sensitive RNA detection method (RT-LAMP) to enable detection of SARS-CoV-2 in the aerosol in quantities comparable to those found for influenza virus during flu season in closed spaces.

Existing high-accuracy methods to detect SARS-CoV-2 require a sample of physiological material (from nose or throat), and hence cannot be used for aerosol virus detection. By contrast, this method allows sampling of virus from the air in closed environments by using an electrostatic precipitation-based air sampler. Also, this method using RT-LAMP retains the high sensitivity of RT-qPCR, but does not require a thermal cycler, which prevents on-site detection. By contrast RT-LAMP is a one-step reaction that can be completed by heating a sample to 65° C.

This system for monitoring and detecting virus present in the air of closed environments can be used broadly in both public spaces and private homes or businesses. The system acts as an early warning of the presence of potentially harmful concentrations of SARS-CoV-2 (or other viruses) in the air. As such, it can trigger an alarm to alert surrounding people that an evacuation or an air-exchange may be required for safety. This system will find application in hospitals, clinics, care-homes, schools, colleges, airports, cruise ships, and other public places where virus can spread.

### Section 4. Process and Portable Device to Extract/Detach Nucleic Acids From Virions Collected on a Metal Probe and Concentrate Them Into Aqueous Solution

The following section of the description provides an example implementation of a process and portable device to extract/detach nucleic acids from virions collected on a metal probe and concentrate them into aqueous solution.

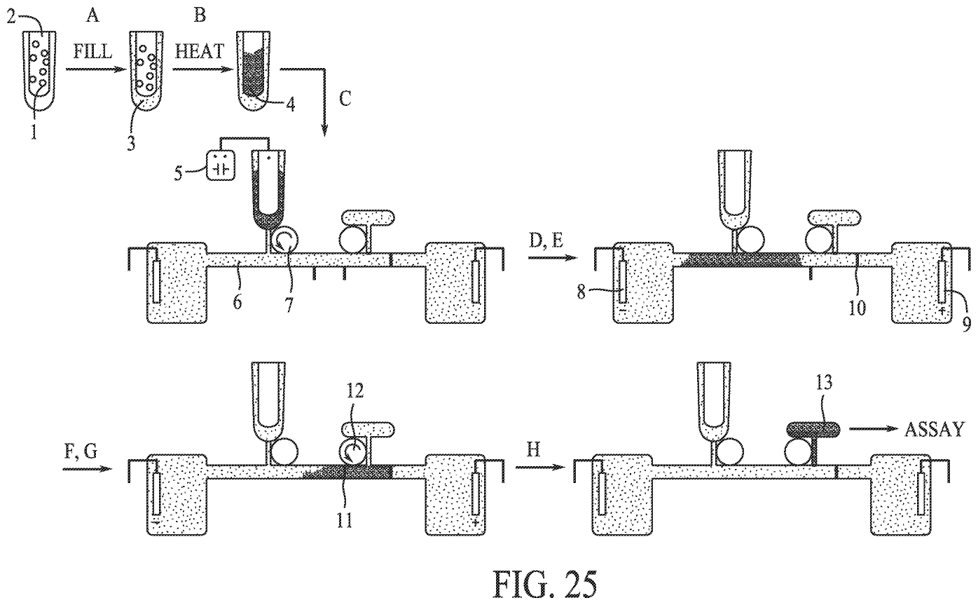

FIG. 25 depicts the process, along with the specific implementing devices, which allow to extract/detach nucleic acids from virions collected on a metal probe and concentrate the nucleic acids into aqueous solution for chemical detection via a biomolecular assay. The illustrated process and devices are portable and can be operated on site. Efficient extraction/detachment and concentration are critical to ensure that we can reach the chemical assay LoD with sufficiently small numbers of viral particles collected on the metal probe.

### Section 4.1. Process for Electrostatic Repulsion of Viral Nucleic Acids From a Collecting Conductive Surface in Aqueous Solvent

FIG. 25A-C shows a method and device to extract RNA from virions collected on a metal probe and disperse them in aqueous solution for downstream processing. This process can extract viral genomic nucleic acid contents (DNA or RNA; RNA will be used as an example here) from intact virions collected on a metal probe.

Nucleic acids obtain a strong negative charge in aqueous solution naturally primarily through the ionization of phosphate groups along the backbone of the polymer. Due to their high negative charge density, nucleic acids can be strongly influenced by electrostatics and electrokinetics.

The process and device presented here is a method for detaching RNA from a collecting conductive surface exploiting the natural negative charge of RNA in solution. When an electrically conductive metal collection surface (e.g., which can be termed probe) becomes negatively charged in aqueous solution, the surface will tend to repel negatively charged species due to its negative potential. The Debye-Hückel theory predicts that negatively charged species are excluded within a distance on the order of a “Debye length”, a factor that depends on the ionic strength of the electrolyte solution. In general, the Debye length can be assumed to be much greater than the distance from the interface at which fluid flow is unachievable due to viscous effects, termed the “no-slip plane”. Thus, electrostatically repelling RNA from the probe-liquid interface pushes RNA to a distance where fluid flow can be employed to remove RNA from the vicinity of the probe.

The electrically conductive metal probe can be supplied with excess negative charge by bringing the metal brought into contact with a conductor containing excess negative charge. This form of charge equilibration can be accomplished using a variety of methods, including a simple circuit where the negative plate of a charged capacitor is isolated and brought into contact with the probe. As a consequence of Gauss' law, negative charges placed on the probe will distribute over the outer surface of the conductor resulting in a negative surface charge density.

Here, a process is presented using the principles of electrostatics to detach RNA from a collector probe, enabling the RNA to be recovered in solution during later steps. The elements of the process are as follows, where each letter corresponds with a step of FIG. 25 and each number represents the item labeled in the drawing. A. Airborne viruses (1) are collected on a metal surface probe (2) via electrostatic precipitation and remain adsorbed to the surface when it is brought into contact with aqueous solvent (3). B. RNA (4) is extracted via heating, causing virions to lyse their genomic content in the vicinity of the probe surface. At this point, RNA naturally ionizes due to its contact with water, gaining negatively charged phosphate groups. C. Using a capacitor (5), a negative surface charge is applied to the conducting probe. Consequently, RNA is repelled from the surface. RNA molecules distribute into the bulk solution, possibly assisted by flow-driven mixing.

### Section 4.2. Process for Charge-Driven Electrical Migration and Concentration of Nucleic Acids for Biomolecular Assays in Aqueous Solvent

Method and device to concentrate, for example, the RNA, into a smaller volume for subsequent biomolecular assays. A process is described for transferring RNA via electrophoresis and increasing the local concentration of RNA via the same effect (see FIG. 25D-H), using principles of electrokinetics and electrostatics.

Charged species existing in an electrostatic field permeating linear, isotropic, conducting aqueous media are subject to electrokinetic effects including electrophoresis. As stated previously, RNA (and other genetic material, such as DNA) obtains a strong negative charge in aqueous solution and can be strongly influenced by electrokinetics. Thus, the application of a uniform electric field in aqueous media containing RNA tends to result in electrophoretic migration of RNA in the direction opposite to the electric field. The same underlying principle causes DNA to migrate in an agarose gel during DNA electrophoresis, an analytical technique in molecular biology.

Here, a process is presented, along with the implementing device, using the principles of free electrophoresis to guide movement of RNA through a detection device and to concentrate the species in a smaller volume for later detection steps. The elements of the process and device are as follows, where each letter corresponds with a step of FIG. 25 and each number represents the item labeled in the drawing. D. Following capture and RNA extraction, RNA molecules (4) are suspended in the aqueous solution used to detach them from the capture device, and this liquid (3) is injected into a capillary (6) via a pump (7). E. Two electrodes (8, 9) present in their own chambers on either end of the capillary are activated with a voltage differential. F. RNA migrates via electrophoresis until it reaches a semipermeable membrane (10), which stops it from traveling further. G. A concentration gradient of RNA develops against the semipermeable membrane. After the gradient has developed, a valve (11) closes to isolate the most concentrated fraction of the RNA. H. RNA-enriched solution is directed via a pump (12) into another chamber (13) where a biomolecular assay is performed.

### Section 5. Portable Device to Detach Aerosol-Loaded Virus From Air-Sampler Probe and Enrich it in Aqueous Solution

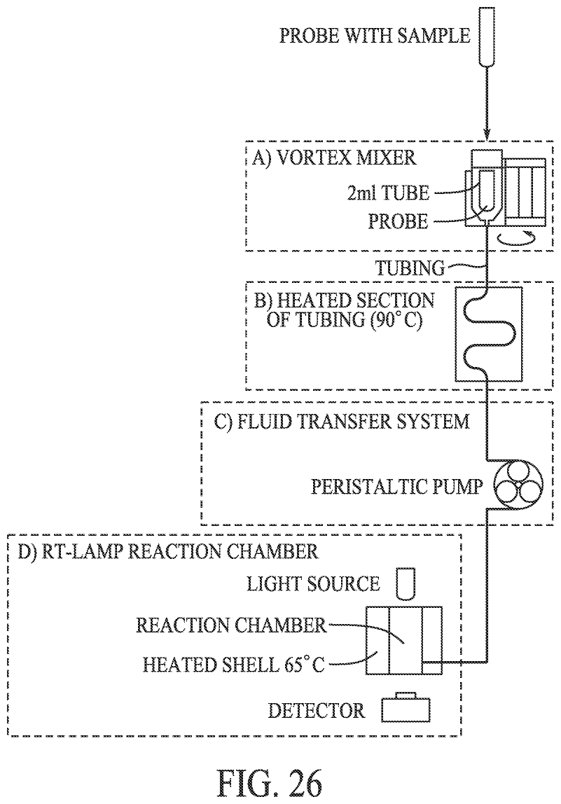

In this section using RNA as an example and shown in FIG. 26 is an automated and portable device is described that accomplishes the steps of (a) detachment/enrichment of the airborne particles from the probe device into aqueous solution; (b) RNA extraction in aqueous solution; (c) fluid transfer into reaction chamber of RNA classification. This system is integrated into a mobile and autonomously moving platform carrying an air sampler. Four components of this example implementation of the device are: a. Miniature vortex mixer (A): The 2-mL tube with the probe device and 50 μL deionized water is vortex mixed to wash off viral particles from the probe device and concentrate them in aqueous solution. b. RNA extraction chamber (B): The fluid transfer system pumps solution from the tube into a heated section of the tubing where it is held for 5 min to release the RNA genome from the viral capsid. c. Fluid transfer system (C): A precise volume of the heat-treated sample is transferred to the RT-LAMP reaction chamber. RT-LAMP nucleic acid detection. As the sample is transferred to the reaction chamber, it is mixed with the RT-LAMP reagents which are present in the reaction chamber. The sample and reagents are incubated at 65° C. via a heated shell. While the sample is incubated, the RT-LAMP chemical reaction occurs and the fluorescence is monitored by the readout system. If the fluorescence signal indicates that the level of viral RNA is over the LoD, the device outputs Yes, otherwise the device outputs No.

The remainder of this section provides details on the design of each component shown in FIG. 26.

### Section 5.1 Miniature Vortex Mixer

In order to detach the collected particles from the probe device, the probe device is placed into a 2 mL tube with deionized water. The tube is translated in a circle about an axis parallel to the axis of the tube, but offset by 2.45 mm at 3200 rpm. This motion is known as vortex mixing, and is typically achieved by bulky laboratory vortex mixer. A scaled down vortex mixer which fits a single 2 mL tube is designed based on the same principles as commercially available vortex mixers.

### Section 5.2 Heated Section of Tubing

To perform the extraction of viral RNA as tested, the solution containing the particles detached from the probe are held at 90° C. for 5 minutes. The proposed method includes is a section of tubing embedded into a heated metal block that is temperature regulated to be at 90° C. The section of tubing inside the heated block is designed such that at least 50 of fluid fits inside the length of tubing within the heated block.

### Section 5.3 Fluid Transfer System

The fluid transfer system serves to transfer fluid between the different parts of the device without manual intervention. The fluid transfer system is designed to be capable of pumping 50 μL of fluid from the vortex mixer (see FIG. 26A) through the depicted tubing into the heated section of tubing (see FIG. 26), and then after the 5 minutes for the viral RNA extraction, transfer a precise volume of fluid from the heated section of tubing to the RT-LAMP reaction chamber (see FIG. 26D). Since all of fluid transfer occurs along a single length of tubing as shown in FIG. 26, a single peristaltic pump can achieve all of the necessary fluid transfer steps. However, if additional precision is desired, other fluid transfer architectures involving pumps and valves are possible, such as an injector with a sample loop, which is commonly used in flow injection analysis.

### Section 5.4 RT-LAMP Reaction Chamber

The RT-LAMP reaction chamber contains the sample and reagents during the RT-LAMP reaction and has integrated heating and fluorescence detection. A heated shell provides a constant heating at 65° C. to conduct RT-LAMP reaction. The heated shell is composed of a metal piece shaped to fit around the reaction chamber with an electric heating control. The geometry of the reaction chamber is designed such that the light source and detector can be used to monitor fluorescence of the contents of the reaction chamber.

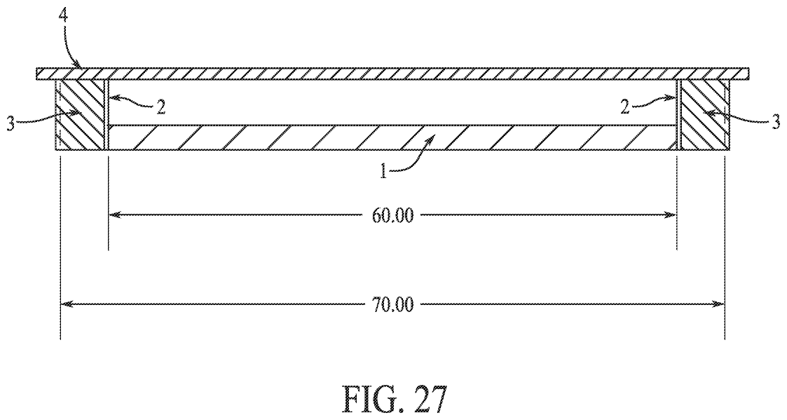

FIG. 27 shows a prototype that was built to demonstrate a proof-of-principle for RNA enrichment via electrophoresis against a semipermeable membrane. The prototype includes: (1) a polydimethylsiloxane (PDMS) chip forming the base and sides of a channel, (2) semipermeable membranes sealing both ends of the channel, (3) 2% agarose gel blocks immediately outside of the membranes to prevent convection, and (4) a glass plate forming the top of the channel. The channel was filled with a solution including either 5.4 or 0.6 ng/μL RNA in 0.125% agarose and Tris-acetate-EDTA (TAE) buffer. The whole apparatus was placed in a standard gel electrophoresis bath containing TAE buffer used to apply an electric field. FIG. 27 shows the dimensions of the prototype measured in millimeters.

The total RNA was extracted from dCas9-transformed E. coli. Before the experiment, a 500 μL aliquot of RNA-containing solution was set aside after homogenization for later analysis. The PDMS chip was filled with 5 mL of homogenous RNA-containing solution by injection using a syringe. The 2% agarose end blocks were added manually to the ends of the chip. The whole device was submerged in the electrophoresis bath and aligned so that the channel was parallel to the direction of the electric field and the membranes perpendicular to the field. A potential difference of 150 V was applied between the electrodes in the electrophoresis bath, creating an electric field of approximately 1500 V/m, and electrophoresis was allowed to proceed for 2 hours. After cessation of electrophoresis, the chip was removed from the electrophoresis bath and 500 μL of solution was removed from the channel by a syringe placed proximally to the membrane nearest to the positive terminal.

The RNA content of the samples collected before and after electrophoresis was quantified by quantitative reverse-transcriptase polymerase chain reaction (qRT-PCR). For each initial RNA concentration tested (5.4 and 0.6 ng/μL), the fold change in RNA concentration was calculated.

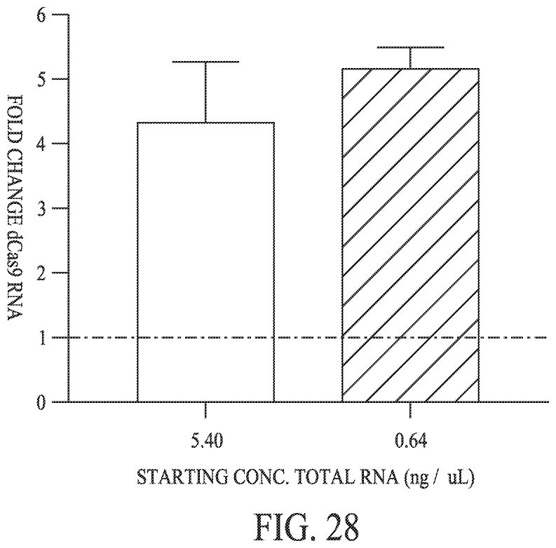

FIG. 28 shows a graph of the fold change of RNA content after membrane-based electrophoretic RNA enrichment in the prototype device. Error bars represent the standard deviation of the fold change across 4 dilutions of the sample.

After performing electrophoresis, a 500 μL enriched fraction was sampled proximal to the positive-terminal membrane, comprising approximately 10% of the volume loaded into the channel. Relative fold changes (“FC”) in dCas9 mRNA concentration between this sample and the starting sample were measured by qRT-PCR (see FIG. 28). A positive FC indicates higher RNA levels after enrichment. A similar degree of enrichment was seen whether 5.4 or 0.6 ng/μL total RNA was used at the beginning of the experiment (FC=4.3 and 5.2 respectively). This experiment indicates that it is possible to concentrate RNA 5-fold or more in a simple prototype with the apparatus and process presented herein.

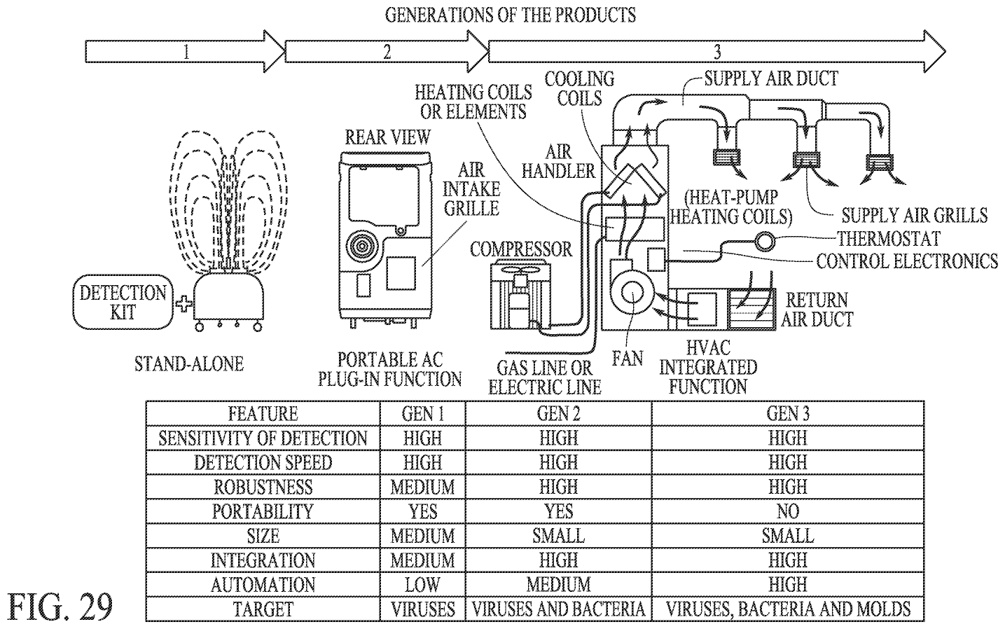

FIG. 29 shows a schematic illustration of multiple different collecting systems. For example, the stand-alone collecting system can be portable and moveable, the portable AC plug-in function can also be portable and can be integrated within a cooling system, and the HVAC integrated function system can be integrated within an HVAC system.

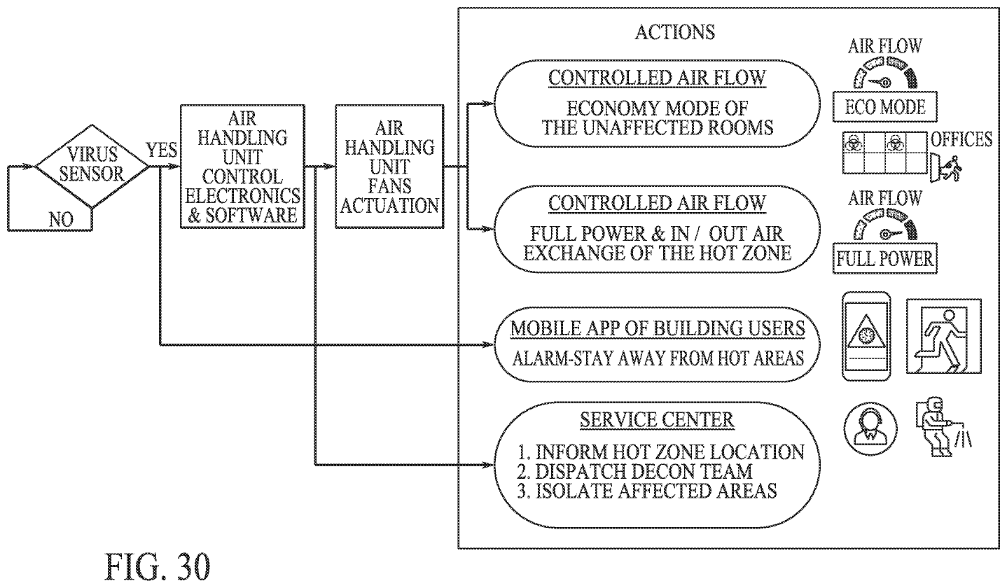

FIG. 30 shows a flowchart of a process for detecting pathogens in the air (e.g., viruses).

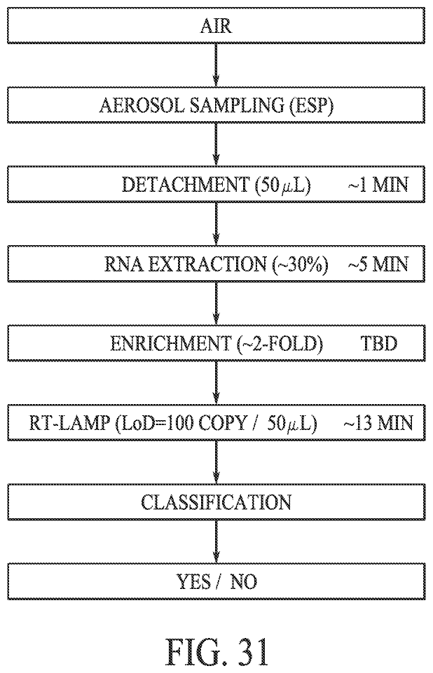

FIG. 31 shows a flowchart of a process for detecting pathogens in the liquid (e.g., viruses).

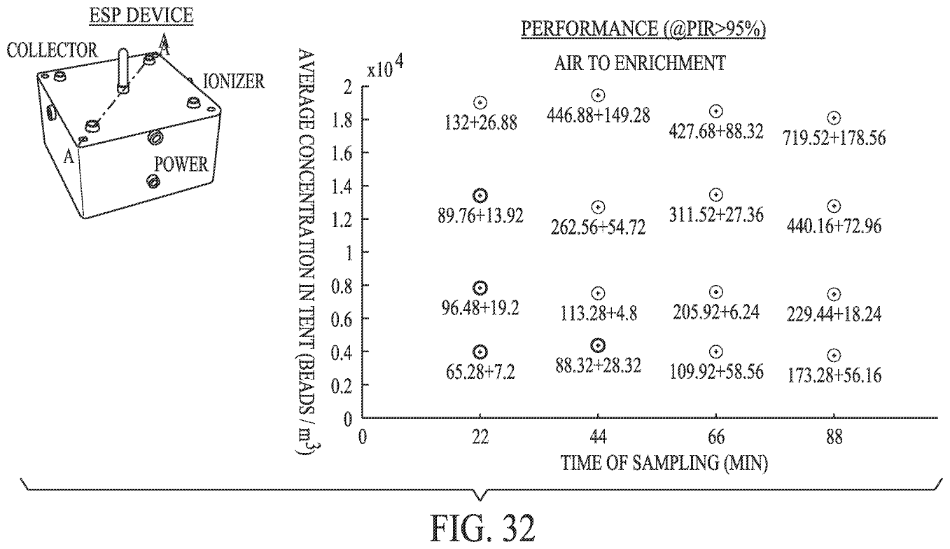

FIG. 32 shows a device and a graph of the performance of the graph.

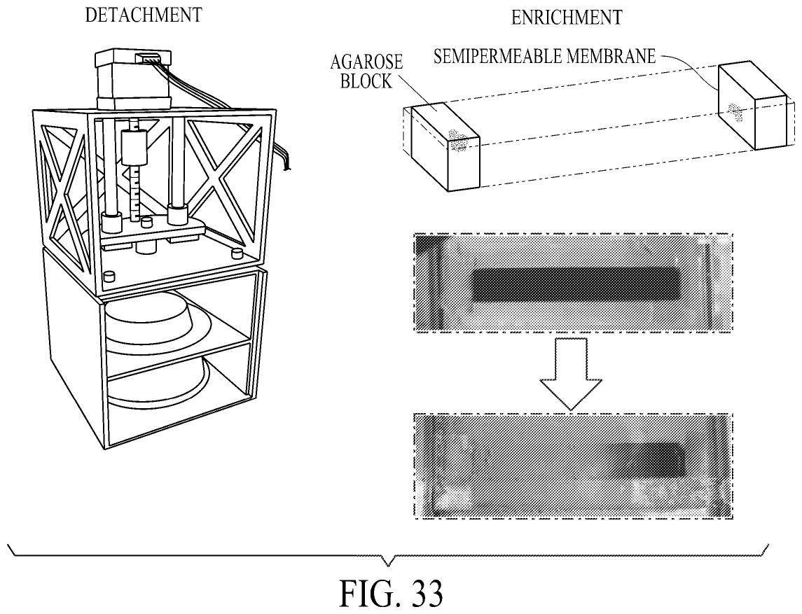

FIG. 33 shows a detachment device and an enrichment device.

The present disclosure has described one or more preferred non-limiting examples, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the accompanying description or illustrated in the accompanying drawings. The disclosure is capable of other non-limiting examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular non-limiting examples or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or non-limiting examples. Further, references to particular rotational or other movements (e.g., counterclockwise rotation) is generally intended as a description only of movement relative a reference frame of a particular example of illustration.

In some non-limiting examples, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, non-limiting examples of the disclosure can be implemented as a set of instructions, tangibly embodied on a non-transitory computer-readable media, such that a processor device can implement the instructions based upon reading the instructions from the computer-readable media. Some non-limiting examples of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular non-limiting examples of the disclosure. Further, in some non-limiting examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as non-limiting examples of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use non-limiting examples of the disclosure. Various modifications to the illustrated examples will be readily apparent to those skilled in the art, and the generic principles herein can be applied to other examples and applications without departing from the principles disclosed herein. Thus, non-limiting examples of the disclosure are not intended to be limited to non-limiting examples shown, but are to be accorded the widest scope consistent with the principles and features disclosed herein and the claims below. The accompanying detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” Further, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of each of A, B, and C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C. In general, the term “or” as used herein only indicates exclusive alternatives (e.g. “one or the other but not both”) when preceded by terms of exclusivity, such as “either,” “one of,” “only one of,” or “exactly one of.”

Various features and advantages of the disclosure are set forth in the following claims.

What is claimed is:

1. A collecting system comprising:
a probe configured to collect pathogens from a surrounding fluid;
an elution chamber containing a liquid solvent and configured to receive the probe to elute the pathogens collected on the probe using the liquid solvent; and
a heater configured to lyse the pathogens to release the genetic material of the pathogens into the liquid solvent.

2. The collecting system of claim 1, further comprising an elution device configured to elute the pathogens collected on the probe into the liquid solvent,
wherein the elution device includes:
a container that defines the elution chamber; and
a heat block in thermal communication with the heater and the elution chamber, the heater being configured to heat the heat block thereby heating the liquid solvent to lyse the pathogens.

3. The collecting system of claim 1, further comprising an elution device configured to elute the pathogens collected on the probe into the liquid solvent, and
wherein the elution device includes at least one of:
a mixer that mixes the liquid solvent and forces the liquid solvent into contact with the probe to elute the pathogens off the probe and into the liquid solvent;
an electrode configured to store charge and repel or attract the pathogens or the genetic material of the pathogens away from the probe and into the liquid solvent; or
a power source in selective electrical communication with the probe, the power source being configured to charge the probe to repel the pathogens or the genetic material of the pathogens away from the probe into the liquid solvent.

4. The collecting system of claim 1, further comprising a concentrator configured to concentrate the genetic material of the pathogens.

5. The collecting system of claim 4, wherein the concentrator includes:
a semi-permeable barrier positioned within a flow path of the genetic material of the pathogens; and
an electrode that is configured to be positively or negatively charged to direct the genetic material of the pathogens towards the semi-permeable barrier to concentrate the genetic material.

6. The collecting system of claim 5, wherein the concentrator includes a first electrode that is configured to be positively charged, and
wherein the concentrator includes a second electrode that is configured to be negatively charged, and
wherein the semipermeable barrier is positioned between the first electrode and the second electrode.

7. The collecting system of claim 1, further comprising at least one of:
a robotic arm that is configured to selectively grasp the probe to place the probe into and out of the elution chamber; or
an actuator that is coupled to the probe, the actuator being configured to be extended to place the probe into the elution chamber or retracted to remove the probe from the elution chamber.

8. The collecting system of claim 1, further comprising a detection system that is configured to detect a presence of the pathogens.

9. The collecting system of claim 8, wherein the detection system includes:
a detection chamber;
a light source optically coupled to the detection chamber;
a photodetector optically coupled to the detection chamber.

10. The collecting system of claim 7, wherein the collecting system is configured to amplify the amount of the genetic material.

11. The collecting system of claim 8, wherein the detection system is configured to detect the presence of a pathogen by detecting the genetic material of the lysed pathogens.

12. The collecting system of claim 11, further comprising a computing device that is configured to:
determine that the pathogen is aerosolizable based on the presence of the pathogens having been detected, the pathogen having been previously unknown of being aerosolizable; and
notify a user based on the determination that the pathogen is aerosolizable.

13. The collecting system of claim 8, wherein the genetic material is amplified without the use of a thermocycler.

14. The collecting system of claim 13, wherein the collecting system does not use a thermocycler.

15. The collecting system of claim 8, further comprising a thermocycler that is configured to cycle the temperature of the liquid solvent and the genetic material to amplify the genetic material.

16. The collecting system of claim 15, further comprising one or more reagents that are configured to implement a PCR on the genetic material to amplify the genetic material.

17. The collecting system of claim 15, wherein at least one of:
the one or more cycles that define the PCR take less than 120 minutes; or
the one or more reagents, the thermocycler, or both are configured to implement a fast PCR on the genetic material to amplify the genetic material, the one or more cycles that define the fast PCR being less than 3 minutes, and
wherein the one or more cycles are at least one of greater than 30 cycles, greater than 35 cycles, or greater than 40 cycles.

18. The collecting system of claim 8, further comprising a computing device in communication with the detection system, and
wherein the computing device is configured to:
cause the light source to emit first light towards the detection chamber, the first light interacting with a molecule that interacts with the genetic material of the pathogens to emit second light;
receive optical data from the photodetector indicative of the second light interacting with the photodetector; and
determine a presence of the pathogen, based on the optical data.

19. The collecting system of claim 18, wherein the collecting system is configured to at least one of:
implement a reverse transcription loop mediated isothermal amplification (RT-LAMP) reaction on the genetic material from the pathogen to amplify the amount of genetic material; or
implement a reverse transcription polymerase chain reaction (“RT-PCR”) on the genetic material from the pathogen to amplify the amount of genetic material.

20. The collecting system of claim 19, further comprising one or more reagents that include:
a primer specific to a corresponding region on the genetic material of the pathogen;
a reverse transcriptase; and
a deoxyribonucleic acid (“DNA”) polymerase.

21. The collecting system of claim 19, further comprising one or more reagents that are configured to implement the RT-LAMP reaction, the one or more reagents being preloaded within the detection chamber.

22. The collecting system of claim 21, wherein the one or more reagents are lyophilized.

23. The collecting system of claim 21, wherein the heater or another heater is configured to heat the genetic material and the one or more reagents to a temperature that is greater than or equal to 65° C.

24. The collecting system of claim 15, wherein the computing device is further configured to:
determine an initial amount of the genetic material in the detection chamber before amplification, based on the optical data; and
determine a concentration of the pathogen in the enclosed volume, based on the initial amount of genetic material and the total volume of the enclosed volume.

25. The collecting system of claim 24, wherein the computing device is further configured to determine the concentration of the pathogen in the enclosed volume, based on the initial amount of genetic material, the total volume of the enclosed volume, and an efficiency ratio of the collection rate of the pathogens in the surrounding volume to the pathogens collected on the probe.

26. The collecting system of claim 9, further comprising a pump that is configured to move the genetic material from the elution chamber and to the detection chamber.

27. The collecting system of claim 1, further comprising a power source in selective electrical communication with the probe,
wherein the probe is in selective electrical communication with an electrical ground,
wherein when the probe is electrically connected to the electrical ground, the probe attracts and collects aerosolized pathogens that have been ionized, and
wherein when the probe is electrically connected to the power source, the power source charges the probe to repel the pathogens or the genetic material of the pathogens away from the probe into the liquid solvent.

28. The collecting system of claim 1, wherein the volume of the liquid solvent is at least one of less than or equal to 250 μL, less than or equal to 150 μL, less than or equal to 100 μL, less than or equal to 50 μL, less than or equal to 25 μL, or less than or equal to 12.5 μL.

29. A method for collecting and analyzing particles, the method comprising:
collecting particles on a probe;
placing the probe into an elution chamber that includes a liquid;
lysing the particles while the probe is positioned within the elution chamber, so that the particles release analytes within the liquid thereby creating analyte solution;
concentrating the analyte solution to create a concentrated analyte solution;
detecting the analyte to detect the presence of the particles.

30. The method of claim 29, wherein lysing the particles includes heating the liquid with the probe positioned within the elution chamber thereby lysing the particles to release the analytes.

31. The method of claim 30, wherein the analyte is charged, and further comprising electrically charging the probe to repel the analyte away from the probe.

32. The method of claim 29, wherein the analyte is charged, and wherein concentrating the analyte includes charging one or more electrodes to cause the analyte to concentrate into the concentrated analyte solution

33. The method of claim 29, wherein the particles are a type of pathogen, and
wherein the analyte is the genetic material of the type of pathogen.

34. The method of claim 29, wherein the type of pathogen is a virus, and
wherein the genetic material is a single stranded RNA.
